(19) United States
(12) Patent Application Publication
(10) Pub. No.: US 2012/0107284 A1
(43) Pub. Date: May 3, 2012

(54) Stem cells for transplantation and methods for production thereof

(76) Inventor: Elena Kozlova, Stockholm (SE)

(21) Appl. No.: 12/308,919

(22) PCT Filed: Jun. 29, 2007
(86) PCT No.: PCT/SE2007/000636

§ 371 Date: Jun. 30, 2009

Related U.S. Application Data
(60) Provisional application No. 60/818,172, filed on Jun. 30, 2006.

(51) Int. Cl. A61K 48/00; A61P 21/00; A61P 3/10; A61P 9/10; A61P 25/00; A61P 9/00

(52) U.S. Cl. 424/93.21

(57) ABSTRACT

A medicament for cell differentiation to alleviate cell and cell-related deficiencies in a mammal is described. The medicament comprises in two or more parts in vitro produced non-activated inducible gene construct(s) capable of expressing transcription factor(s), and optionally additional suppressor(s) or activator(s) of expression of said transcription factor(s), in a cell for transfection, such as a stem cell, and separate exogenous inducer(s) for in vivo expression, such as tetracycline, streptogramin and macrolide. Examples of the cell and cell-related deficiencies are traumatic injuries to the brain and spinal cord, neurodegenerative disorders, stroke, demyelinating disorders, neuropathic pain disorders, diabetes, myocardial infarction, skeletal muscle disorders. Further, a delivery system for delivery of the medicament to a mammal as well as a method of treating cell and cell-related deficiencies in a mammal, are described.

### FIELD OF INVENTION

The present invention relates to controlled differentiation of stem cells, and in particular to a medicament in two or more parts for cell differentiation to alleviate cell and cell-related deficiencies in a mammal, comprising in vitro produced non-activated inducible gene construct(s) capable of expressing transcription factor(s), and optionally additional suppressor(s) or activator(s) of expression of said transcription factor(s), in a cell for transfection, and separate exogenous inducer(s) for in vivo expression.

### BACKGROUND

Transplantation of stem cells is an attractive strategy for cell replacement in trauma and a wide range of disorders which are associated with cell/tissue degeneration. This cell replacement approach is particularly relevant for disorders of the brain and spinal cord. A major issue in stem cell transplantation is to control their differentiation to the desired type of cells. According to state of the art, stem cells are prepared for differentiated into their type of cell, before transplantation, but their differentiation in vivo is unpredictable.

Recently, a number of systems have been developed, which allow the regulated expression of multiple genes in an independent and sequential manner at the time of need (reviewed in Weber and Fussenegger, 2006). One of these systems is the tetracycline-regulated gene expression system (Tet-system; Gossen and Bujard, 1992, 2002), which is well characterized and has been extensively improved and adapted in recent years for maximum performance in different cell lines, and also been tested for its usefulness as a gene therapeutic strategy (Goverdhana et al, 2005). The Tet-system has also been introduced in animals, making it possible to prepare stem cells from transgenic embryos or adult mice in which the components of the Tet-system have already been incorporated (Ludwig et al, 2004, Sonntag et al, 2004; Rubinchik et al, 2005).

### Gene Regulating Systems

Differentiation of specific types of cells in the nervous system follows an intrinsic genetic program in concert with external factors (Guillemot, rev 2007). The intrinsic genetic program consists of TFs, which have to be switched on/off in a specific sequence and at specific time points. The principle transcriptional events, which regulate neuronal differentiation, are shown in FIG. 1.

Pro-neural genes, expressed in early embryonic development, segregate populations of neural precursor cells. The induction of pro-neuronal genes identifies a neuronal precursor population, which is subsequently diversified by specific TFs to a variety of neuronal subtypes. The TFs, determining the differentiation of neural precursor cells to fully functional glial cells or neurons, are now known for many neuronal subtypes (See section “Applications”). This information enables directing of the differentiation of neural stem cells to fully functional neurons or glial cells of the desired type.

The Tet system is a conditional regulatory system of prokaryotic origin with inducible promoter system which has been adapted for use in mammalian cells (Gossen and Bujard, 1992, 2002). The original Tet system consists of two components, the tetracycline response element (TRE), and the activator system. TRE is composed of seven repeats of a specific-binding site (tetO) for the transcription activator placed immediately upstream of minimal CMV promoter. The activator encodes a transactivator hybrid protein (tTA) composed of the tetracycline repressor (tetR) fused to the herpes simplex virus (HSV) transactivation domain from VP16. Expression of a gene inserted downstream of the tetO/minimal CMV promoter in the response element is highly dependent on tTA which binds tetO sequences through its TetR domain and recruits positively acting cellular transcription factors through its VP16 domain. Tetracycline binds to the transactivator protein, causing it to dissociate from the tetO/minimal CMV promoter, thereby switching off transcription of the “target” gene.

The original Tet system has subsequently undergone numerous improvements, modifications and expansions (reviewed in Weber and Fussenegger, 2006). As a result of improvements, the possibility of low levels of gene transcription when the Tet system is inactivated has been largely eliminated. Modifications of the activating system allows the system to either switch target gene transcription off (tTA;TetOff) or on (rtTA, TetOn) (FIG. 2). Further addition of modified activating systems offer numerous alternatives to tetracycline or its derivatives as activators of gene silencing/transcription.

Transgenic research has demonstrated that there are specific key TFs, which when expressed ectopically drive differentiation of immature precursor/stem cells towards more mature stages and eventually to full maturation

Through either random integration or transient transfection a construct consisting of a tissue specific or “regional-cell specific” promoter, an rtTA, TRE and TF(s) of choice are introduced into the stem cells. When doxycycline (DOX) is added, rtTA binds the TRE and induces the expression of transgenic TF(s). Alternatively, stable transfection of rtTA under expression control of either a “regional” TF promoter for the specific stem cell type (in accordance with the example of the invention, in the Sox10 locus of transgenic mice) (FIG. 3) or a constitutively active promoter like the EF-1α promoter (Gopalkrishnan et al., 1999) Is established. Thereafter, an additional transfection introduces the TRE-inducible TF(s). These temporally separated transgenes can be delivered to the cell in any order or simultaneously. Transfection of stem cells may be done through electroporation with linearized DNA or by lipofectamine plasmid transfection(s).

The Tet system is widely used for inducible expression control of transgenes in cell lines and organisms (Gossen and Bujard, 2002; Berens and Hillen, 2003; Toniatti et al., 2004). It allows complex regulatory setups to be implemented due to numerous modifications of the individual regulatory components. To be able to express the two target genes in a sequential, independent manner using Tet-system requires two Tet-based transcription factors with the following properties: They must bind different effectors, recognize different DNA binding sites and not form mixed dimers. Exclusive DNA-binding specificities for tet operator variants have been described (Krueger, Berens, and Hillen, in preparation). TetR mutants specifically recognizing dox (Urlinger et al, 2000) or cmt3 (Henssler et al, 2004) have been published. Corresponding transregulators from two natural sequence variants that do not form heterodimers exist (Schnappinger et al, 1998).

To activate independently three genes, an additional system under pharmacological transgene expression control has to be added. Possible additions to the Tet-controlled system are the streptogram in—(PIP-OFF, PIP-ON) or macrolide-controlled (E-OFF, E-ON) regulatory systems developed by the Fussenegger group (reviewed by Weber and Fussenegger, 2006). These systems follow the concept underlying the Tet system and the components are essentially the same, except for their inducer- and DNA-binding moieties (Corbel and Rossi, 2002). They are compatible with each other and can be used in combination for differential and independent expression of multiple target genes (reviewed in Weber and Fussenegger, 2006). Recently appeared technologies also based on volatile effectors, such as acetaldehyde, which can be effective in non-toxic concentrations (Weber et al, 2004). This development adds further potential flexibility for inducible gene regulation.

However, as mentioned, in the state of the art stem cells are differentiated into their type of cell, before transplantation.

### DESCRIPTION OF THE INVENTION

The present invention is based upon the finding that stem cells can be differentiated into the desired type of cell, after transplantation.

Thus, according to the present invention, stem cells are supplied with a gene construct, and subsequently activated in vivo, for differentiation to the target type of cell. This solves a long-felt need of supplying stem cells that differentiate into the right type of cell, and survive.

The invention demonstrates the feasibility of exogenously and conditionally regulated gene expression in stem cells for induction of differentiation to a desired type of neuron or glial cell following transplantation. This is demonstrated in a transgenic cell system in vitro and in vivo for sequential exogenous expression of transcription factors (TFs) that are crucial for implementing/guiding/directing the differentiation of stem cells to fully functional neurons or glial cells of the desired type.

The invention provides production of desired types of neurons or glial cells from stem cells, which are stably or transiently transfected with at least one key transcription factor, the expression of which is under pharmacological control of a heterologous regulatory system. Such a control system enables regulation of the differentiation of transplanted stem cells to the desired type of neuron or glial cells after transplantation.

According to one aspect of the invention, there is provided a medicament in two or more parts for cell differentiation to alleviate cell and cell-related deficiencies in a mammal, comprising in vitro produced non-activated inducible gene construct(s) capable of expressing transcription factor(s), and optionally additional suppressor(s) or activator(s) of expression of said transcription factor(s), in a cell for transfection, and separate exogenous inducer(s) for in vivo expression.

According to one embodiment of the invention, the cell for transfection is chosen from the group consisting of regional stem cells, embryonic stem (ES) cells, neural crest stem cells, neural stem cells from brain and spinal cord, mesenchymal stem cells, endothelial stem cells, endodermal stem cells.

According to another embodiment of the invention, the transcription factor(s) used are chosen from the group consisting of embryonic stem cell transcription factors NANOG, OCT3/4, and Sox2; neural crest stem cell transcription factors Brn3a, FoxD3, GATA-3, Hand2, Mash1, Miff, Nanog, Ngn1/2, Oct-4, Pax3, Phox2a/b, Runx1/3, Slug, Sox4/8/9/10/11; neural stem cells from brain and spinal cord transcription factors Brn3a, Cash1, Cdx2, Dbx1/2, Dlx1/2, Ebf1, Emx1/2, En1/2, ER81, Evx1, Foxg1, Foxp2, Gbx2, Gli2/3, Gsh1/2, Hb9, Hest/5, Hoxb4/9, Id1/3, Islet1/2, Lhx1/2/5/6/7, LIM1/2/3, Lmx1a, Mash1, Math1, MNR2, Msx1, NeuroD, Ngn1/2/3, Nkx2.1/2, Nkx×6.1/2, Olig1/2/3, Otx1/2, Pax3/5/6/7/8, Phox2b, Pitx3, Prox1, Pff1, ROR, Sim1, Sox10, Tbr2, Tlx-3, Trb1; mesenchymal stem cell transcription factors C/EBP-alpha, Dlx5, Fli1, Gata2/3/4, Gli3, KROX20, Msx1/2, Myf5, MyoD, Oct-4, Pax3/6/7, PPAR-gamma, Runx2, Rex-1, Sox2/9/10; endothelial stem cell transcription factors ARNT, ELF-1, EPAS, Ets1, Fra1, GATA2/3, gridlock, HIF-1alpha, HOXD3, LMO2, NERF-2, Prox-1, Runx1, Scl, Sox2, Sp1/3, TBP, Vezf1, YY1; endodermal stem cell transcription factors Beta2/NeuroD, FoxD3, Hex, Hnf3/6, Hixb9, Islet1, MafA, Meis2, Ngn3, Nkx2.2, Nkx6.1, Oct-4, Pax4/6, Pbx1, Pdx1. Use of these transcription factors and their role in cell differentiation is documented, and makes up part of the state of the art. The skilled person easily realizes that these and other transcription factors may be used in the framework provided by the invention.

According to yet another embodiment of the invention, the cell and cell-related deficiencies to be treated in a mammal is a disease chosen from the group consisting of traumatic injuries to the brain and spinal cord, neurodegenerative disorders, stroke, demyelinating disorders, neuropathic pain disorders, diabetes, myocardial infarction, skeletal muscle disorders.

According to still another embodiment of the invention, the inducible gene construct(s) comprise(s) conditional and compatible transcription control systems which are able to provide simultaneous or independent activity of transgene(s) to regulate stem cell differentiation in response to exogenous inducer(s).

Another embodiment of the invention provides that the exogenous inducer(s) for use in the invention is (are) chosen from the group consisting of the medicaments tetracycline, streptogramin and macrolide. However, there may be other than the mentioned inducers that are useful for exogenous gene regulation according to the present invention.

Another aspect of the invention provides a delivery system for delivery of a medicament in two or more parts to a mammal, comprising as a first part non-activated inducible gene construct(s) capable of expressing transcription factor(s) and optionally other construct(s) containing additional suppressor(s) or activator(s) of expression of said transcription factor(s), in a cell, and as a second part exogenous inducer(s) of expression, the delivery of the second part being after or simultaneously with the delivery of the first part.

Still another aspect of the invention provides a method of treating cell and cell-related deficiencies in a mammal, comprising the consecutive steps:

(a) transfection of cells with non-activated inducible gene construct(s) capable of expressing transcription factor(s), and optionally additional suppressor(s) or activator(s) of expression of said transcription factor(s), in a cell;
(b) transplantation of said cells to said mammal, and
(c) activation of said inducible gene construct(s) by administration of exogenous inducer(s) to said mammal for expression of said transcription factors in cells of said mammal.

An embodiment of this last aspect provides that the cells for transfection are chosen from the group consisting of regional stem cells, embryonic stem (ES) cells, neural crest stem cells, neural stem cells from brain and spinal cord, mesenchymal stem cells, endothelial stem cells, endodermal stem cells; and the transcription factors are chosen from the group consisting of embryonic stem cell transcription factors NANOG, OCT3/4, and Sox2; neural crest stem cell transcription factors Brn3a, FoxD3, GATA-3, Hand2, Mash1, Miff, Nanog, Ngn1/2, Oct-4, Pax3, Phox2a/b, Runx1/3, Slug, Sox4/8/9/10/11; neural stem cells from brain and spinal cord transcription factors Brn3a, Cash1, Cdx2, Dbx1/2, Dlx1/2, Ebf1, Emx1/2, En1/2, ER81, Evx1, Foxg1, Foxp2, Gbx2, Gli2/3, Gsh1/2, Hb9, Hest/5, Hoxb4/9, Id1/3, Islet1/2, Lhx1/2/5/6/7, LIM1/2/3, Lmx1a, Mash1, Math1, MNR2, Msx1, NeuroD, Ngn1/2/3, Nkx2.1/2, Nkx6.1/2, Olig1/2/3, Otx1/2, Pax3/5/6/7/8, Phox2b, Pitx3, Prox1, Ptf1, ROR, Sim1, Sox10, Tbr2, Tlx-3, Trb1; mesenchymal stem cell transcription factors C/EBP-alpha, Dlx5, Fli1, Gata2/3/4, Gli3, KROX20, Msx1/2, Myf5, MyoD, Oct-4, Pax3/6/7, PPAR-gamma, Runx2, Rex-1, Sox2/9/10; endothelial stem cell transcription factors ARNT, ELF-1, EPAS, Ets1, Fra1, GATA2/3, gridlock, HIF-1alpha, HOXD3, LMO2, NERF-2, Prox-1, Runx1, Scl, Sox2, Sp1/3, TBP, Vezf1, YY1; endodermal stem cell transcription factors Beta2/NeuroD, FoxD3, Hex, Hnf3/6, Hlxb9, Islet1, MafA, Meis2, Ngn3, Nkx2.2, Nkx6.1, Oct-4, Pax4/6, Pbx1, Pdx1.

According to one embodiment of the invention, principles of pharmacological transgene control systems for gene expression, exemplified by the Tet-system, are used for activating the expression of TFs, which are critical for the differentiation of specific stem cells to the desired type of neurons or glial cells.

According to the invention, small molecule regulator(s) are used, including but not limited to tetracycline, its derivatives or analogues which can be applied at any predefined time point, as well as transactivators like rtTA, tTA or any other pharmacological components (stably or ectopically transfected to the cell) able to bind the chimeric promoter, consisting of specific operators placed adjacent to the minimal promoter (Pmin) which activates expression of the gene of interest. When all necessary components have been delivered to the stem cells, the expression of the target genes(s) can be controlled with the effector(s) and the subsequently induced transcription factor expression(s) will lead to differentiation to the desired cell type in vitro and in vivo.

This embodiment of the invention is applicable to any stem cell type, for which the sequential activation of transcription factors (TFs) or other intracellular molecules critical for the desired cell differentiation has been established.

According to one embodiment of the invention, the TFs Sox10 and Aml1/Runx1 are combined in murine neural crest stem cells (NCSCs), whereby the Tet-system is used for activation of Aml1/Runx1 expression in these cells to guide them to a specific subtype of sensory neurons.

According to embodiments of the present invention,

a) sensory nociceptive neurons are conditionally produced by implementing the Tet-system to neural crest stem cells (NCSCs) in vitro, as well as following transplantation, and
b) Schwann cells are conditionally produced by implementing the Tet-system to NCSCs in vitro, as well as following transplantation.

In one embodiment of the invention, use is made of a, DNA encoding a reverse tetracycline transcription activation unit (rtTA), a DNA construct consisting of TREbi (a chimeric promoter, containing a specific heptameric repeat of the Tet-operator (tetO2) placed adjacent to two divergently oriented minimal promoters (Pmin and Pmin*) and a gene of interest, e.g. Aml1/Runx1, which upon activation of rtTA by doxycycline (DOX) will be expressed in parallel with a green/yellow fluorescent protein (eGFP/eYFP) reporter gene. The ectopic expression of the long isofom of Aml1/Runx1 (Aml1B/Runx1) in NCSCs will induce the differentiation of a specific type of dorsal root ganglion nociceptor neurons, and the ectopic expression of the short isoform, Aml1A/Runx1, will prevent neuronal differentiation and therefore presumably induce the differentiation of Schwann cells.

According to another embodiment, a dual Tet-system for regulated differentiation of stem cells to nociceptors. The cells will be transitory or stably transfected with rtTA, cTA, Ngn2 and Aml1/Runx1 containing plasmids and will, following independent activation of Ngn2 and Aml1/Runx1 expression in vitro and in vivo, guide the differentiation of NCSCs to nociceptor neuron subtype.

According to still another embodiment of the current invention, it is demonstrated that by conditionally regulating the expression of key TFs, guiding of differentiation of stem cells to the specific type of cells of interest following transplantation is enabled. For this purpose, neural crest stem cells were prepared, giving rise to many types of cells, including dorsal root ganglion (DRG) neurons, and their differentiation controlled.

According to yet another embodiment, the expression of several genes is regulated independently, thus increasing the possibilities to generate different types of desired cells from a variety of stem cells, incuding embryonic stem cells by sequential activation of molecules, which guide stem cells to mature and become fully functional.

According to one embodiment of the invention, transgenic mice with regulatable Tet-systems for the controlled exogenous activation of key TFs in stem cells leading to their differentiation to neurons or glial cells of the desired type and function, are used.

### Applications

The present invention may be used in the following non-limiting examples of applications.

Listed below are several applications for the drug regulatable induction of stem cell differentiation applicable to disorders of the nervous system and sensory organs, as well as to other non-neural type of cells, such as insulin-producing beta-cells, cardiac myocytes and skeletal myocytes. As mentioned, this list is intended to serve as examples, without limitations, of different applications using this approach. There are several sources of stem cells for the implementation of drug regulatable differentiation, including embryonic stem (ES) cells (Choong and Rao, 2007), neural crest stem cells from the enteric nervous system (Heanue and Pachnis, 2007), or from hair follicles (Hoffman, 2007), embryonic, fetal or adult neural stem cells (Schwartz et al, 2006; Hsu et al, 2007), bone marrow stem cells (Hermann et al, 2006), and stem cells from adipose tissue (Parker and Katz, 2006).

In the example applications, the source for obtaining the respective regionally specified embryonic neural stem cells in mice is indicated. After regional stem cell preparation the cells can be transfected with tetracycline-responsive constructs encoding transcription factors (TFs) specific for the respective desired cell type differentiation. This approach can help to find the right combination of TFs to guide different stem cell types to the specific differentiated cell type. In one example, use is made of regional stem cells, neural crest stem cells (NCSCs), specifically expressing the neural crest stem cell transcription factor Sox10 the genomic locus of which the rtTA gene was knocked into. To these cells are delivered tetracycline-responsive elements (TRE) controlling the expression of the sensory neuron diversification TF Aml1/Runx1. Moreover, examples of already tested tissue/area specific promoters are provided, which were used in different applications in transgenic mice research. These data can provide a basis for future design of functional Tet-system vectors for tissue/region specific stem cell regulation.

A) In brain injury and stroke stem cells can exert neuroprotective effects, promote intrinsic repair potential, and replace specific populations of neurons (Longhi et al, 2005; Bliss et al, 2007; Dobkin, 2007). The following stem cell sources and TFs are relevant (see also Molyneaux et al, 2007 for review):

GABAergic cortical neurons: Stem cells from E12.5-13.5 ventricular and subventricular layers of the median ganglionic eminence, expressing Nkx2.1, and subsequently Mash1 and Dlx1 (Bellion and Min, 2005; Cobos et al, 2005; et al, 2007).
Glutamateralc cortical neurons: Stem cells from E11.5-12.5 dorsal telencephalon, expressing Pax6-Trb2-NeuroD and Tbr1 (Bellion and Main, 2005; Hevner et al, 2006; Guillemot, 2007).
Cortical oligodendrocytes: Stem cells from E11.5 ventricular and subventricular zone of the ventral forebrain, expressing Sox10, followed by Olig2, and Mash 1 (Parras et al, 2007).
Cortical astrocytes: Stem cells from E11.5-12.5 dorsal telencephalon, expressing Sox9, and thereafter Id1/3 followed by Hes1/5 (Ross et al, 2003; Kageyama et al, 2005).

The Tet-system has been tested in the forebrain, where regulators were successfully placed under control of the alphaCaMKII promoter (Uchida et al, 2006; Mansuy et al, 1998)

B) Transplantation of mesencephalic dopaminergic neurons have, for a long time, been advocated as an attractive source for a treatment strategy in Parkinson's disease (PD). Stem cells are harvested from E12.5-13.5 ventral midbrain, expressing Neurogenin2 (Andersson et al, 2006; Kele et al, 2006), thereafter Nurr1 (Andersson et al, 2007), and subsequently Mash 1 (Park et al, 2006). The nuclear TF Nurr1 is involved in the development and maintenance of the midbrain dopaminergic (DA) neuronal phenotype (Jankovic et al, 2005). The effect of Nurr1 during embryonic stem (ES) cell differentiation was tested using the ROSA26-engineered Tet-inducible ES cell line J1-rtTA.

There is a well established correlation between olfactory symptoms and PD (Berendse and Ponsen, 2006). This provides a possible link between PD and adult neural stem cells, which are continuously produced in the subventricular zone (SVC) of the lateral ventricle, and migrate to the olfactory bulb where they give rise to interneurons. Prior to their maturation, SVZ stem cells express nestin. A nestin-tTA transgenic mouse has been produced and the functionality of the Tet-system in SVZ stem cells was verified (Beech et al, 2004).

Glial cell line-derived neurotrophic factor (GDNF) has been shown to protect and restore DA neurons in injury models and is being evaluated for the treatment of Parkinson's disease (Wu and Frucht, 2005). The tetracycline-dependent transcription activator (tTA)/tTA-responsive promoter system for overexpression of GDNF has been used in transgenic mice (Kholodilov et al, 2004).

C) Cell replacement therapy in Alzheimer's disease is a highly complex problem given that many neuronal systems are involved. However, the transplantation of stem cells, differentiated to influence the disease progression is of great potential interest (Sugaya et al, 2006). The functionality of the Tet-system has been tested in animal models of Alzheimer's disease (Engel et al, 2006; Khlistunova et al, 2006).

D) Multiple sclerosis (MS) is characterized by focal loss of myelin (demyelination), leading to block of impulse propagation, and symptoms, that depend on which pathway(s) are affected. The disease process also involves degeneration of the axons themselves, possibly as a result of increased vulnerability in the absence of the insulating myelin. Whereas functional improvement occurs in the earlier phases of the disease, the intrinsic repair potential eventually becomes exhausted. Production of oligodendrocytes or oligodendrocyte precursors, which are able to develop to myelin forming cells, can be used to replace damaged cells in MS as well as in other central demyelinating disorders (Keyoung and Goldman, 2007). A regional source for this purpose are stem cells from the E13.5 ventral spinal cord, expressing Sox10, followed by Olig2 or Nkx2.2, and Mash1 (Sugimori et al, 2007), or the E11.5 ventral telencephalon, expressing Sox10, followed by Olig2, and Mash1 (Parras et al, 2007).

E) Epilepsy is characterized by an exceptionally low threshold for excitation of synapses in specific forebrain region(s). A rational way to raise the threshold and thereby eliminate epileptic seizures or markedly reduce their frequency is to raise the level of the inhibitory neurotransmitter GABA (cf Castillo et al, 2006). To generate GABAergic interneurons, stem cells are harvested from E12.5-13.5 ventricular and subventricular layers of the median ganglionic eminence, expressing Nkx2.1, and subsequently Mash1 and Dlx1 (Bellion and Métin, 2005; Cobos et al, 2005; Poitras et al, 2007).

F) In spinal cord injury neural stem cells can exert neuroprotective effects, promote intrinsic repair potential, and, finally, replace specific populations of neurons (Pfeifer et al, 2006; Thuret et al, 2006). The following stem cell sources and TFs are relevant:

Spinal motor neurons: Stem cells from E9.5-10.5 ventral spinal cord, expressing Olig2, and subsequently Ngn2 and Mash1 (Sugimori et al, 2007).
Dorsal horn interneurons: Stem cells from E9.5-10.5 dorsal spinal cord, expressing Pax2, and subsequently Pax5 or Pax8, and Lhx1 or Lhx5 (Pillai et al, 2007).
Spinal oligodendrocytes: Stem cells from E13.5 ventral spinal cord, expressing Sox10, followed by Olig2 or Nkx2.2, and subsequently Mash1 (Sugimori et al, 2007).
Spinal astrocytes: Stem cells from E12.5-13.5 dorsal spinal cord, expressing Sox9 (Stolt et al, 2003), and subsequently Id1 and Hes 1 or Hes 5 (Kageyama et al, 2005; Sugimori et al, 2007).

G) In motor neuron disease, notably amyotrophic lateral sclerosis (ALS), transplantation of appropriate stem cells to the spinal cord/brainstem can counteract further loss of motor neurons, replace already lost ones, and restore neuromuscular connections. Recent evidence indicates that dysfunctional astrocytes contribute significantly to the disease progression in ALS. An alternative/additional cell replacement action may therefore be to transplant stem cells, which are competent to differentiate to spinal cord astrocytes.

Spinal motor neurons: Stem cells from the E9.5-10.5 ventral spinal cord, expressing Olig2, and subsequently Ngn2 and Mash1 (Sugimori et al, 2007)
Spinal astrocytes: Stem cells from the E12.5-13.5 dorsal spinal cord, expressing Sox9 (Stolt et al, 2003), and subsequently Id1 and Hes 1 or Hes 5 (Kageyama et al, 2005; Sugimori et al, 2007).
Brainstem motor neurons: Stem cells are harvested from E9.5 rhombomeres 2-4, expressing Mash 1, followed by Phox2b, and thereafter Mat1+Math3 (Ohsawa et al, 2005).

The Tet-system has been tested in the motor neuron-like cell line NSC-34, in which the regulatory protein tTA was stably transfected. The cell line NSC-34-tTA was co-transfected with the cDNA of the human Cu/Zn superoxide dismutase, a gene of interest in ALS, which was cloned into pBI-EGFP, downstream of the tetracycline-responsive bidirectional promoter. This plasmid was transiently transfected into NSC-34-tTA40, and the functionality of bidirectional transcription was verified by determining the expression of enhanced green fluorescent protein and of human Cu/Zn superoxide dismutase (Babetto et al, 2005).

H) Neuropathic pain is a common and therapeutically problematic condition, which often emerges after stroke or trauma to the peripheral or central nervous system. In general, the condition is caused by a dysbalance between excitatory and inhibitory mechanisms in spinal or supraspinal circuits mediating various sensations of pain. A rational longterm treatment strategy is to raise the inhibitory level of the circuitry mediating the painful experiences by transplanting stem cells that develop to GABAergic neurons (cf. Lee et al, 2007; Wolfe et al, 2007). For spinal cord circuitry, stem cells are harvested from the E9.5-10.5 dorsal spinal cord, expressing Pax2, and subsequently Pax5 or Pax8, and Lhx1 or Lhx5 (Pillai et al, 2007). For supraspinal circuitry, stem cells are harvested from E12.5-13.5 ventricular and subventricular layers of the median ganglionic eminence, expressing Nkx2.1, and subsequently Mash1 and Dlx1 (Bellion and Métin, 2005; Cobos et al, 2005; Poitras et al, 2007).

I) Transplantation of stem cells to the eye is an attractive strategy, particularly with regard to loss of retinal photoreceptors but also in disorders causing loss of retinal ganglion cells (Young, 2005; Harvey et al, 2006 MacLaren et al, 2006). To replace retinal photoreceptors stem cells are harvested from E12.5-13.5, or E16.5 optic cup, expressing Otx2, thereafter Neurogenin2, NeuroD and Mash1. To replace retinal gangl ion cells, stem cells are harvested from E16.5 optic cup, expressing Pax6, thereafter Math5 and NSCL1 (Yan et al, 2005; Harada et al, 2007). For inducible gene expression in retinal ganglion cells, Thy1 and ckit promoters were used to direct expression of a second-generation reverse tetracycline transactivator (rtTA2S-M2) (Kerrison et al, 2005).

J) Hearing loss, often associated with tinnitus and balance problems is a common and seriously disabling condition. The most common cause of these symptoms is loss and/or dysfunction of hair cells of the inner ear. Loss of auditory sensory neurons is another cause of hearing loss and associated problems, and is caused by primary disease or secondarily to loss of hair cells. Stem cell implantation to the inner ear can serve to counteract further cell destruction, and restore lost auditory function (Hu and Ulfendahl, 2006). To replace hair cells, the specific source of stem cells are Sox2 expressing cells in the otic E9.5 placode. These differentiate to mature hair cells by expressing Neurogenin1 followed by Atoh (Fritzsch et al, 2006). To replace lost auditory sensory neurons, stem cells from the E9.5 otic placode, expressing Sox2, subsequently differentiate to cochlear sensory neurons by expressing Neurogenin1 and NeuroD1 (Fritzsch et al, 2006).

K) Allotransplantation of newly generated muscles for restoration of lost or wasted muscle tissue (Morgan, 2005; Saini A et al, 2006). Recently the role of the transcription factors Pax3 and Pax7 in regulating differentiation of specific progenitor cells and in regulating their entry into the skeletal muscle differentiation program was demonstrated (Buckingham and Relaix, 2006).

A modified muscle creatine kinase (MCK) promoter was recently developed to generate a skeletal muscle-specific, doxycycline (DOX) controlled over-expression system in mice. The codon optimized reverse tetracycline transactivator (rtTA) was placed under control of a skeletal muscle-specific version of the mouse MCK promoter. Transgenic mice containing this construct expressed rtTA almost exclusively in skeletal muscles (Grill et al, 2003). A human cytokeratin 18 expression cassette to drive epithelium-specific expression of the reverse tetracycline transactivator (rtTA) was tested in the skin (Ye et al, 2001). In another example, tTA and rTA were expressed by the bovine keratin 5 promoter to demonstrate conditional expression of genes in the mouse epidermis (Diamond et al, 2000).

The functionality of the Tet-system was tested on hair follicles by using the bovine keratin 5 promoter to drive expression of the tetracycline-regulated transactivators tTA and rTA, and a constitutively active mutant of TGFbeta1 linked to the tetO target sequence for the transactivator (Liu et al, 2001).

L) An attractive source for cell replacement treatment in myocardial infarct is mesenchymal stem cells (Fukuda K and Fujita J, 2005). The Tet-system using rtTA driven by the cardiac α-myosin heavy chain promoter was successfully tested in transgenic mice (Valencik and McDonald, 2001; Tumbull et al, 2006; McCloskey et al, 2005; Valencia et al, 2000).

M) Stem cell transplantation for the replacement of insulin producing β cells is an attractive therapy for longterm treatment of diabetes (Gangara m-Panday et al, 2007). Recently the differentation of embryonic stem cells to insulin-producing cells was achieved in vitro (Schroeder et al, 2006). Generation of insulin-secreting cells from human embryonic stem cells was also demonstrated in vitro (Baharvand et al, 2006). Stem cells that give rise to the definitive endocrine pancreas emerge at E13.5, and express a series of key TFs, including Pdx1 and Hnf6, followed by Ngn-3, Beta2/NeuroD nd Pax4 (Ackermann and Gannon, 2007). The tetracycline-regulated transactivator (tTA(off)) has been tested in beta cell differentiation by replacement of the coding region of the endogenous Pdx1 gene. Expression of the transgene-encoded Pdx1 rescues the Pdx1-null phenotype; the pancreata of these mice develop and function normally (Holland et al, 2002).

### Example

### Tet-System Mediated Differentiation of Sensory Neuron Subtype In Vitro and In Vivo.

In accordance with the invention, neural crest stem cells expressing sox10-rtTA are used and show that the sequential activation of the transcription factor (TF) Aml1/Runx1 in these cells leads to their differentiation to specific type of neurons. This example illustrates the potential of achieving desired stem cell differentiation by using sequential activation of key TF expression.

Regulated Differentiation of Neural Crest Stem Cells with a Single Tet-System In Vitro Introduction

The neural crest cells generate a variety of sensory and autonomic neurons as well as glial cells of the peripheral nervous system, pericytes and smooth muscle cells of the vascular system, including the cardiac outflow tract, chromaffin cells (endocrine cells of the adrenal gland) and most pigment cells. In addition, neural crest cells originating from the developing head give rise to connective tissue of the cranial muscles and chondrocytes, osteoblasts and odontoblasts, and components of the craniofacial skeleton.

Many regulatory signals have been described that promote the formation of particular cell fates in migratory and postmigratory neural crest stem cells (NCSCs), including signals specifying the differentiation of sensory neurons from neural crest cells (for review see e.g. Sommer, 2001). The sequential expression of transcription factors Sox10, Neurogenin (Ngn) 1/2 and Aml1/Runx1 identify the critical steps in the differentiation of NCSCs to a subtype of dorsal root ganglion neurons.

### Sox Proteins

Sox proteins belong to the family of high-mobility group (HMG-box) transcription factors which are already expressed in the neuroectoderm and function as neural competence factors (Ma et al, 1999). Sox9 expression occurs transiently in the premigratory neural crest before expression of Sox10, which continues in migrating NCSCs (Hong and Saint-Jeannet, 2005).

Enteric nervous system: The Sox10 signalling pathway is required during the development of the enteric nervous system and of melanocytes, and its role in Waardenburg-Hirschsprung disease (hypopigmentation, deafness and absence of enteric ganglia) is well established (Stanchina et al, 2006). Sox10 has a crucial role in the maintenance of multi-lineage enteric nervous system progenitors (Bondurand et al, 2006).
Hair follicles: The presence of pluripotent NCSCs in the adult mammalian hair follicle was recently demonstrated (Sieber-Blum et al, 2004). The bulge region of the adult hair follicle contains the niches for both epithelial and melanocyte stem cells. Recent evidence indicates that the development of melanocyte stem cells is controlled by a complex network of transcription factors, including Sox10 (Sommer, 2005).
Boundary cap: The boundary cap (b) is a transient structure during embryonic development at the entry/exit zones of dorsal and ventral spinal nerve roots, respectively. bNCSCs constitute a common source of cells for functionally diverse types of neurons, as a single bNCSC can give rise to several types of nociceptive and thermoreceptive sensory neurons (Hjerling-Leffler et al, 2005).

Thus, Sox10 is expressed in all listed types of NCSCs and delays neuronal differentiation in these cells, both in vitro and in vivo and preserves glial and neuronal potential from extinction by lineage commitment signals. Thus, Sox 10 plays a role in maintenance of stem/progenitor cells. At the postmigratory stage when NCSCs reach their final destination in the developing PNS, cells that maintain Sox10 expression differentiate to glial cells whereas in cells which differentiate to neurons Sox10 expression is turned off (Kim et al, 2003; Inoue et al, 2004).

### Runx1 Transcription Factors

The later expressed Runx TFs determine critical steps in DRG neuron differentiation (Marmigère and Emfors, 2007). Runx3 is expressed at high levels in developing cranial and dorsal root ganglia proprioceptive neurons, and acts to diversify an Ngn1-independent neuronal subpopulation and is a tyrosine kinase receptor (Trk) C neuron specific transcription factor. Runx3-deficient mice develop severe limb ataxia due to disruption of monosynaptic connectivity between dorsal root afferents and motoneurons as a result of loss of DRG proprioceptive neurons (Levanon et al, 2002).

Aml1/Runx1 is expressed in the TrkA subpopulation of DRG neurons during embryogenesis (Levanon et al, 2001). In this population, Aml1/Runx1 acts as a transcriptional activator and is necessary first for specific differentiation and later for survival of nociceptors. One direct role of Aml1/Runx1 in the differentiation of the nociceptor subclass is the induction of TrkA expression. In the absence of Aml1/Runx1, TrkA is not expressed and the neurons die by apoptosis (Marmigère et al, 2006). Postnatally, Aml1/Runx1 becomes restricted to nociceptors marked by expression of the neurotrophin receptor Ret, suppresses the emergence of a peptidergic phenotype, and controls the lamina-specific innervation pattern of nociceptive afferents in the spinal cord (Chen et al, 2006).

Thus, Runx3 and Aml1/Runx1 regulate the emergence of subpopulation specific DRG neuron characters (Marmigère and Emfors, 2007). bNCSCs give rise to nociceptive and thermoreceptive sensory neuron subtypes (Hjerling-Leffler et al, 2005), which express Aml1/Runx1 during the period of their neuronal specification. These stem cells with established TFs sequence for their differentiation are the stem cells of choice in the example provided.

### Neurogenin 1/Neurogenin2

The generation of DRG neurons is controlled in progenitor cells also by the combinatorial activities of Neurogenin (Ngn) 1 and 2, two proneural TFs of the basic helix-loop-helix class, which follow Sox10 TF expression. The combined absence of Ngn1/2 results in a complete lack of DRG neurons (Ma et al, 1999). Most TrkC⁺ and TrkB⁺ DRG neurons appear to be derived from Ngn2 precursors, whereas Ngn1 is required for generation of the majority of TrkA⁺ afferents (Ma et al., 1999).

### Guiding DRG Neuron Differentiation by Sequential Activation of Transcription Factors

The established sequence of TFs determining the differentiation of NCSCs to DRG neurons provides a possibility to genetically regulate the differentiation of NCSCs. It is demonstrated that the Tet-system mediated the activation of Aml1/Runx1 expression to control the differentiation of DRG neurons from NCSCs harvested from transgenic mice harbouring the knock-in rtTA in the Sox10 locus. Sox10-rtTA mice are bred with TRE-Aml1/Runx1 mice and stem cells prepared from their offsprings. Alternatively, NCSCS from Sox10-rtTA mice are transfected with TRE-Aml1/Runx1. In both cases, treatment of the NCSCs with DOX will induce Aml1/Runx1 expression and, as a result, influence their differentiation to nociceptive DRG neurons.

Regulated Differentiation of Neural Crest Stem Cells with a Single Tet-System In Vivo

### The Dorsal Root Injury Model for Cell Replacement and Neural Repair

Injuries to the dorsal roots cause permanent loss of sensory functions due to the inability of dorsal root axons to regenerate into the spinal cord. As a result of these injuries patients also often suffer intractable neuropathic pain (Terzis et al, 2004). The basis for the permanent loss of sensory functions is the inability of injured sensory neurons to overcome the non-permissive environment at the junction of the peripheral (PNS) and central nervous system (CNS) in the dorsal root (Silver and Miller, 2004). The centrally directed process of DRGs starts off in the PNS and enters the CNS at the dorsal root transitional zone (DRTZ), which develops around birth as a conical extension of central tissue projection to the dorsal root. After its development, injured dorsal root axons re-grow without interruption in the PNS part of the root, but cease to grow when they encounter the DRTZ. The dorsal root injury paradigm is therefore an attractive system for studying differentiation and repair processes after cell replacement.

Success in repairing injury induced sensory disconnection between the peripheral and central nervous system by transplanting embryonic human DRGs to the dorsal root ganglion cavity of adult recipient rats has previously been shown. Axons of transplanted DRGs were able to grow through the DRTZ and restore functional synaptic connections in the spinal cord (Levinsson et al, 2000).

A method to transplant neural stem/progenitor cells (NSPCs) as neurospheres and dissociated cells to the DRG cavity after removal of the DRG and to the intact DRG, respectively, was recently developed. It was shown that NSPCs can survive; some of them differentiated to neurons, whereas most of transplanted cells differentiated to peripheral type glial cells both in the DRG cavity and in the intact DRG (Brännvall et al, 2006). The DRG injury paradigm is an attractive model for exploring processes underlying differentiation of transplanted stem cells. This model is used in accordance with the invention to demonstrate the differentiation of transplanted Sox10-rtTA bNCSC neurospheres by Tet-system mediated activation of Aml1/Runx1.

### DESCRIPTION OF THE FIGURES

FIG. 1. Schematic illustration of the sequence of events leading from a multipotent stem cells to a differentiated neuron. Transcription factors of the bHLH family induce the emergence of proneural progenitor cells. As a result of subsequently expressed specific transcription factors in combination with Notch signalling, these cells become committed to a pan-neuronal fate. Additional key transcription factors determine the final diversification of neuronal subtypes.

FIG. 2. Schematic overview of the tetracycline-regulated gene expression system used in this study. The target gene (TG), is under the control of the TetR binding sequence, tetO, linked to a minimal promoter (Pmin). In the tTA-dependent expression system (A), the tTA protein (circles) binds to the tetO promoter and activates transcription of the TG gene. Incorporation of doxycycline (DOX) into the medium prevents tTA from binding to tetO, resulting in the absence of TG expression. The reverse tTA system (B) takes advantage of a reverse tetracycline transactivator, rtTA (circles) that binds to the tetO promoter when DOX is present. In this case, rtTA activates the target gene only in the presence of DOX. (Modified from Urlinger et al, 2000).

FIG. 3. Schematic illustration of the tetracycline-regulated gene expression system used in the example in accordance with the invention. In the presence of DOX, the reverse transcriptase transactivator (rtTA) in the Sox10 locus activates the Aml1/Runx1 gene, and as a result the transcription factor Aml1/Runx1 is expressed.

FIG. 4. The plasmid used for producing eYFP-TRE_{bl}Aml1A/Runx1 mice.

FIG. 5. The plasmid used for producing Rosa26-rtTA/HPRT-Aml1A/Runx1-IRES-eGFP mice.

FIG. 6. The eYFP-TRE_{bl}-Aml1B/Runx1 plasmid used (SEQ ID NO: 5) transfection with the Aml1B/Runx1 long isoform of neurospheres prepared from boundary cap neural crest stem cells (bNCSCs) of E11 Sox10-rtTA embryos.

FIG. 7. Schematic illustration of the generation of reporter constructs for transfection. The constructs were made in the Edinburgh laboratory on the basis of the bidirectional pBl-L vector (see Figure). The luciferase reporter was substituted with the enhanced yellow fluorescent protein (eYFP). The reporter constructs encode bTRP driving both Aml1/Runx1 cDNA and eYFP in opposite directions. The eTFP construct allow non-invasive control of gene expression in live tissues, providing a tool for monitoring and sorting out Aml1/Runx1 overexpressing cells (by reviewed Naylor, 1999)

Two responsive constructs with different isoforms of Aml1/Runx1 (A and B) were generated. Restriction sites marked with asterisks had generated 5′ overhangs which were filled in after digestion. A restriction site marked with an asterisk generated 3′ overhangs which were removed. Gray arrow marks an FRT site; black triangle denotes a IoxP site; blue left-right arrow marks bTRP.

FIG. 8. Abnormalities in offspring of pregnant Sox10-rtTA mice which had been mated with TRE-Aml1/Runx1 males and treated with DOX during pregnancy days 7-8 and 11.5-14. Pups with the genotype [Sox10-rtTA+TRE-Aml1/Runx1] are smaller than normal and display pigment defects (upper left), megacolon (lower left), and a reduced size of dorsal root ganglia (upper and lower right).

FIG. 9. The number of dorsal root ganglion neurons is reduced compared to control (1) by about 50% in [Sox10-rtTA+TRE-Aml1/Runx1] pups from Sox10-rtTA mice, which hade been mated with TRE-Aml1/Runx1 males, and treated with DOX during pregnancy days 7-8 and 11.5-14 (2).

FIG. 10. Immunohistochemical labelling of Sox10 in dorsal root ganglia (DRG) of an 11 days old Sox10-rtTA embryo (left), and in neurospheres derived from E11 Sox10-rtTA embryos (right).

FIG. 11. Expression of transcription factors measured with quantitative RT-PCR in neurospheres cultured for three weeks after preparation from boundary cap neural crest stem cells of E11 Sox10-rtTA embryos. The neurospheres express high levels of Sox10, moderate levels of neurogenin (Ngn) 2, and low levels of Aml1/Runx1 and Runx3.

FIG. 12. Expression of transcription factors measured with quantitative RT-PCR in neurospheres prepared from boundary cap neural crest stem cells of E11 Sox10-rtTA embryos, and transfected with a plasmid containing Aml1 B/Runx1 long isoform. Twenty-four hours after transfection, neurospheres were treated with DOX, and transcription factor expression analyzed 24 h later. Transfection and DOX treatment induced a marked up-regulation of neurogenin (Ngn) 1, a transcription factor expressed in a cohort of neuronal progenitors, from which nociceptive sensory neurons differentiate.

FIG. 13. Expression of transcription factors in the control experiment to that shown in FIG. 12. Neurospheres were prepared and cultured in the same way, but were not transfected with Aml1B/Runx1 long isoform. Under these conditions, there is up-regulation of neurogenin (Ngn) 2 and Runx3, which are characteristic of proprioceptive, rather than nociceptive sensory neurons.

FIG. 14. Overview of experimental paradigms. Left: Transgenic mice experiment based on breading Sox10-rtTA mice with TRE-Aml1/Runx1 mice and preparation of the stem cells from their embryos. Middle: Transgenic mice and transfection experiment based on preparation of stem cells from Sox10-rtTA mice and subsequent transfection with TRE-Aml1/Runx1. Right: Planned transfection of stem cells with dual Tet-system.

FIG. 15 left. DOX mediated induction of the Aml1A/Runx1 reporter gene, enhanced yellow fluorescent protein (eYFP) in neurospheres prepared from boundary cap neural crest stem cells from E11 embryos of Sox10-rtTA pregnant mice, which had been mated with eYFP-TRE-Aml1A/Runx1 males. The genotype of the cells in the neurospheres was determined by prior embryo genotyping. After verification of Sox10 expression in the neurospheres, DOX was given to one group of neurospheres for 24 h. Non-treated neurospheres served as control. eYFP expression was induced only in DOX treated cultures.

FIG. 15 right. DOX mediated induction of the Aml1 B/Runx1 long isoform reporter gene, eYFP in neurospheres prepa red from neural crest stem cells from E11 embryos of Sox10-rtTA pregnant mice and transfected with eYFP-TRE-Aml1B/Runx1 long isoform. After verification of Sox10 expression in the neurospheres, DOX was given to one group of neurospheres for 24 h. Non-treated neurospheres served as control. eYFP expression was induced only in DOX treated cultures.

FIG. 16. Influence of Aml1A/Runx1 (short isoform) and Aml1 B/Runx1 (long isoform) on differentiation of dorsal root ganglion (DRG) neurons in culture. Treatment with DOX induced extensive differentiation with the characteristic morphology of DRG neurons in E11 Sox10-rtTA neurospheres transfected with Aml1 B/Runx1 (long isoform) (left), whereas no such neurons were found in neurospheres prepared from Sox10-rtTA+TRE-Aml1A/Runx1 (short isoform) mice (right).

FIG. 17. Dorsal root ganglion neurons with a characteristic morphology in neurospheres prepared from boundary cap neural crest stem cells from E11 Sox10-rtTA embryos and transfected with Aml1B/Runx1 long isoform. Neurospheres were cultured for five days in the presence of DOX, thereafter fixed in phosphate buffered formalin-picric acid, and immunolabelled with antibodies to beta-tubulin, a marker for neurons.

FIG. 18. Glial cells in dissociated neurospheres. Neurospheres were prepared from boundary cap neural crest stem cells from E11 [Sox10-rtTA+Aml1/Runx1] embryos. Cultures were treated with DOX for 5 days, thereafter fixed with phosphate buffered formalin-picric acid and immunolabelled with antibodies to Mts1/S100A4 (left), a marker for peripheral glial cells, and Sox10 (right).

FIG. 19. Differences in cellular differentiation in neurospheres harbouring Aml1A/Runx1 (left) and Aml1B/Runx1 (long isoform; right). Neurospheres were prepared from boundary cap neural crest stem cells from E11 [Sox10-rtTA+Aml1A/Runx1] embryos (left) and E11 Sox10-rtTA embryos and transfected with Aml1B/Runx1 (long isoform; right). Cultures were treated with DOX for 2 days, thereafter fixed with phosphate buffered formalin-picric acid and immunolabelled with antibodies to neurons (beta-tubulin) or glial cells (glial fibrillary acidic protein). Neurospheres in which Aml1A/Runx1 is induced preferentially differentiate to glial cells (left), whereas neurospheres in which Aml1B/Runx1 (long isoform) is induced preferentially differentiate to neurons (right).

FIG. 20. DOX induced up-regulation of enhanced green fluorescent protein (eYFP) in transplanted neurospheres. Neurospheres prepared from E11 Sox10-rtTA embryos and transfected with Aml1B/Runx1 (long isoform) were cultured for two hours in the presence of the nuclear dye Hoechst (1:2000, Invitrogen) and collected for transplantation. Adult mice of the C3H strain were anaesthetized with a mixture of Xylazine and Ketamine, and xylocain injected into the area of surgery. After a skin incision and gentle removal of part of the lumbar back muscle, the laminae of lumbar (L) vertebrae 4 and 5 were removed on the left side to expose dorsal root ganglia L4 and L5. The ganglia were removed, a pellet of neurospheres was placed in each ensuing cavity, and covered with a thin layer of gelfoam (Spongostan®). Muscles and skin were sutured in layers, and the animals allowed to recover. Animals were given daily injection of Cyclosporin A (Sandimmun®) and doxycycline in their drinking water (0.3% docycyclin hyacate, 5% sucrose). Three weeks after transplantation, the animals were re-anaesthetized and perfused via the left ventricle with 0.9% saline followed by phosphate buffered formalin-picric acid. The transplant site was removed, postfixed for four hours, cryoprotected overnight in 15% phosphate buffered sucrose and sectioned on a cryostat at 8 μm. Transplanted cells were identified by the presence of Hoechst (left), as well as by DOX mediated up-regulation of eYFP (right).

FIG. 21. Neuronal differentiation in transplanted neurospheres. Experiments were carried out as described in FIG. 19, post-transplantation survival time was three weeks. Sections of the transplant were immunolabelled with antibodies to the neuronal marker beta-tubulin. Transplanted neural crest stem cells were identified by the presence of Hoechst (right). About 10% of Hoechst-positive cells expressed beta-tubulin (left).

FIG. 22. Beta-tubulin immunolabelled neurons in transplanted neurospheres, adjacent to part of recipient dorsal root ganglion. Experiments were carried out as described in FIG. 19. Cells labelled with the neuronal marker beta-tubulin in transplanted neurospheres were smaller than dorsal root ganglion neurons of the adult recipients.

FIG. 23. Doxycycline (DOX) mediated induction of binding of Griffonia Simplicifolia Agglutinin isolectin B4 (B4). Experiments were carried out as described in FIG. 19. Animals were given DOX in their drinking water for two weeks after transplantation and then perfused. Sections through the transplant site were labelled with rhodamine-conjugated B4, a marker for a subtype of nociceptive dorsal root ganglion neurons. A fraction of Hoechst labelled cells were also labelled with B4. The neurospheres contained Sox10-rtTA and transfected with Aml1B/Runx1 (long isoform).

FIG. 24. Doxycycline (DOX) mediated induction of binding of Griffonia Simplicifolia Agglutinin isolectin B4 (B4). Experiments were carried out as described in FIG. 19, except that transplanted neurospheres were prepared from E11 sox10rtTA and Rosa26-rtTA embryos and transfected with Aml1B/Runx1 (long isoform). Animals were given DOX for 3 weeks and then perfused. Transplanted cells are identified by Hoechst labelling (right). Transplanted cells show strong binding of rhodamine-conjugated B4 (left). A fraction of Hoechst labelled cells were also labelled with B4.

FIG. 25 A: Regulatory system. Transregulators are shown schematically with their designations and their functional domains for DNA-binding (small circles), effector-binding and dimerisation (large ellipses) or transcription activation (black circles). Transcription factor binding sites on the DNA (TRE) are named and represented by a box. Cognate pairs are marked identically. Target gene expression is indicated by an arrow, non-expression by a broken arrow.

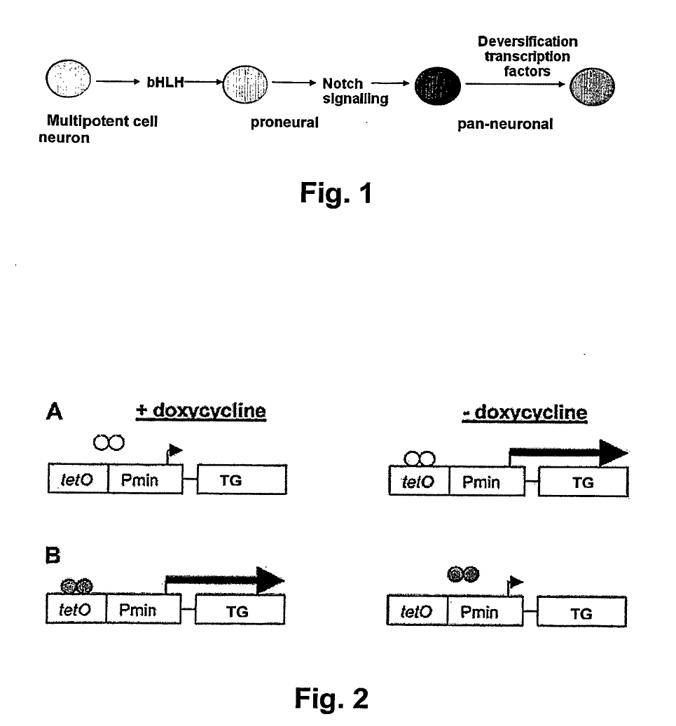

B: Regulatory properties. HeLa cells (HLRF33/1141) were cotransfected with two plasmids encoding the transregulators cTA2^{D}-5 and rtTA2^{s}-M2_{4C5G}. Reporter gene expression was scored after 24 h incubation with 1 μg/ml of either cmt3 or dox or both. Luciferase activities were determined from cell extracts and represent the means of triplicate samples with standard deviations given in arbitrary light units (ALU; defined as relative light units per mg of total cell protein corrected for transfection efficiency). Expression of Renilla luciferase controlled by rtTA2^{s}-M2_{4C5G} is increased about 1000-fold and only in the presence of dox (columns 3 and 4). Addition of cmt3 leads to a ˜100-fold decrease in firefly luciferase activity, controlled by cTA2^{D}-5 (columns 2 and 4), while expression of firefly luciferase is only slightly, less than 2-fold, reduced in the presence of dox. These findings show that it is possible to achieve exclusive expression of either firefly luciferase (equivalent to Runx1 activation in this experiment) in the absence of effectors and of Renilla luciferase (corresponding to Ngn2 activation in this experiment) in the presence of both cmt3 and dox. The regulatory system is thus functional and can be used for target gene expression in NCSCs.

FIG. 26. Plasmid rtTA2S-M2-4C5G (SEQ ID NO: 7) is designed for planned transfection to create a dual Tet-system in stem cells for inducing their differentiation to DRG neurons. The reading head of the DNA recognizes the 4C5G sequence on the operator.

FIG. 27. Plasmid cTA2D-5 (SEQ ID NO: 8) is designed for planned transfection to create a dual Tet-system in stem cells for inducing the differentiation to DRG neurons. The reading head of the DNA recognizes the wild type sequence on the operator.

FIG. 28. Plasmid pWHE1141+hNgn2 (SEQ ID NO: 9) is designed for planned transfection to create a dual Tet-system in stem cells for inducing the differentiation to DRG neurons.

The plasmid contains the tetracycline responsive cassette with the Ngn2 gene. The activation by DOX will induce binding of rtTA2S-M2-4C5G to the cassette, leading to Ngn2 expression.

FIG. 29. Plasmid for transfection with short Aml1A/Runx1 (SEQ ID NO: 6) isoform is designed for planned transfection and which will be used as a control plasmid for the dual Tet-system experiment.

### METHODS USED IN THE FOLLOWING EXPERIMENTS

### Animals

Mice with the following genotypes were used:

Sox10-rtTA2^{s}-M2 (Ludwig et al, 2004)

Rosa26-rtTA

eYFP-TRE-Aml1A/Runx1 (random transfection)

Rosa26-rtTA HPRT-Aml1A/Runx1-IRES-eGFP

These mice were used for breeding to obtain dorsal root ganglia (DRGs) from 11 day old embryos (E11). From these DRGs neural crest stem cell (NCSC) cultures were prepared.

### Genotyping

Males and females were genotyped before breeding, as well as each individual embryo from which DRGs for stem cell cultures were collected. Primers for genotyping mice with the Aml1/Runx1 gene or Rosa26-rtTA were designed to specific parts of the different transgenic constructs (see below).

### DNA Preparation

### Materials

Eppendorfs 1.5 ml (Axygen, Catalog #MCT-150-A)

Pipettes and pipette tips

### Reagents

Taq-Polymerase (Fermentas Life Science; cat# EP0404)

100 bp DNA ladder, GeneRuler (Fermentas Life Science; SM0243)

dNTP (Bioline, cat# B10-39043)

Agarose (Bioline, cat# B10-41026)

DNA

Na Acetate 3M

Ethanol 96.5%

Ethanol 70%

Proteinase K 16 mg/ml

1M Tris pH 8.0

0.5M EDTA pH 8.0

10% SDS

Isopropanol

Ethidium bromide

Preparation of Lysis-Buffer (100 ml)

5 ml 1M Tris pH

20 ml 0.5M EDTA pH 8.0

5 ml 10% SDS

70 ml H₂O

### Equipment

Thermomixer 1400 RPM

Centrifuge 1-14 (Sigma, Labex instrument AB)

### Procedure

Genomic DNA is prepared from biopsies from ear (newborn) or tail (embryo)
Add 250 μl lysis-buffer and 10 μl proteinase K to the tail biopsy
Shake 1-2 h at 55° C. and 1400 RPM in the thermomixer until the tissue is dissolved
Let the sample cool to RT (about 21-22° C.)
Add 0.8 Vol Isopropanol (200 μl) to precipitate the DNA. Shake the tubes until you see the DNA precipitate
Centrifuge for 15 min at 14000 RPM
Remove supernatant and wash the pellet with 70% Ethanol (500 μl)
Centrifuge for 5 min at 14000 RPM
Remove supernatant carefully and let the pellet dry at RT
Add 1000 μl H₂O to the pellet, shake it at 55° C. and centrifuge at 1400 RPM in the thermo mixer until the DNA is dissolved (takes about 1h)
Take 1 μl DNA solution as template for genotyping by PCR

### PCR

### Materials

Eppendorfs 0.2 ml (Axygen, Catalog #PCR-02-C)

Pipettes and pipette tips

### Equipment

Automated thermal Cycler (Gene Amp PCR system 9700)

Thermomixer 1400 RPM

Centrifuge 1-14 (Sigma, Labex instrument AB)

Gel electrophoresis

UV-board

### Primers for Genotypinq

A. Sox10-rtTA2^{s}-M2 mice (Ludwig et al, 2004)
Sox10 cDNA Acc. no. BC018551

Common upper primer located 425-445 bp upstream of the Sox10 start codon:

(SEQ ID NO: 10)
Sox10-F: 5′-CTAGGCTGTCAGAGCAGACGA-3′

Specific lower primer for Sox10 located 13-33 bp downstream of the start codon:

(SEQ ID NO: 11)
Sox10-R: 5′CTCCACCTCTGATAGGTCTTG-3′

Product length of Sox10-F/Sox10-R: 478 bp

Specific lower primer for rtTA2^{s}-M2 located 153-173 bp downstream of the start codon:

(SEQ ID NO: 12)
rtTA2^{S}-M2-R: 5′-CTCGATTGGCAGGGCATCGAG-3′

(marked in bold and highlighted in cyan) Product length Sox10-F/rtTA2^{s}-M2-R: 618 bp
B. Rosa26-rtTA mice (Gossen et al, 1995; Wutz & Jaenisch, 2000; Kyba et al, 2002; http://www.zmbh.uni-heidelberg.de/Bujard/rtTA/pUHD172-1neo.html)

Sequence pUHD172-1contains rtTA-nls that was used to generate Rosa26-rtTA mice

rtTA-nls expression cassette (in blue): EcoRI 766, BamHI 1810 (cds: 775-1800 with ATG and TAG marked in bold; nls marked in italics (Figure)

(SEQ ID NO: 13)
rtTA-F CGCAATGGAGCAAAAGTACA
(SEQ ID NO: 14)
rtTA-R CCTCGATGGTAGACCCGTAA
(complementary: TTACGGGTCTACCATCGAGG (SEQ ID NO:
15))

Product size: 391 bp; primers in bold—
→both primers bind to the rtTA sequence

According the Kyba et at (2002), Wutz' and Jaenisch (2000) rtTA originates from the pUHD172-1 neo plasmid (Gossen et al., 1995). The plasmid sequence is available on http://www.zmbh.uni-heidelberg.de/Bujard/rtTA/pUHD172-1neo.html

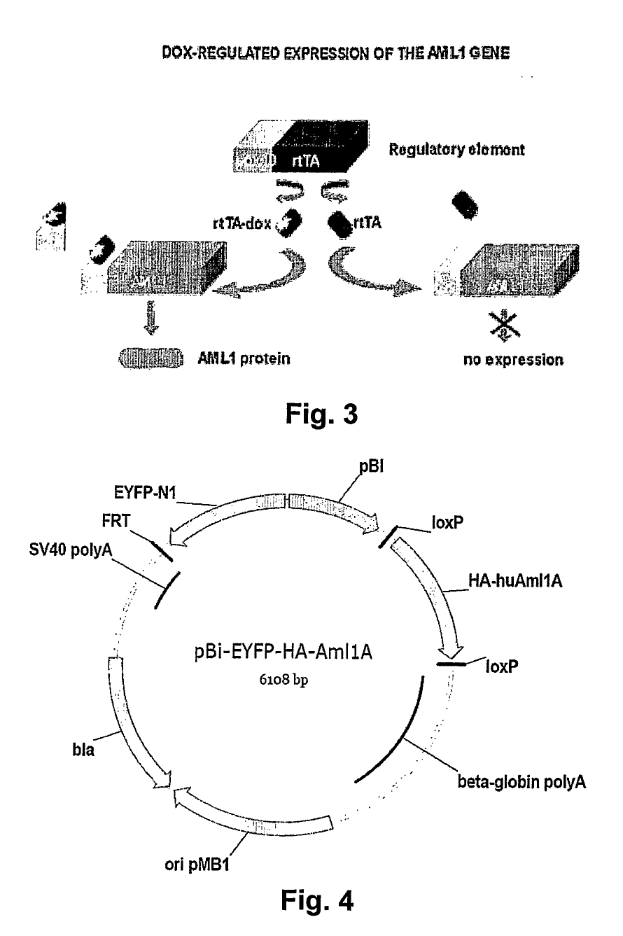

C. eYFP-TRE_{bl}Aml1A/Runx1 mice (random transfection) (Kitabayashi, Tokyo; Likhovitskaia and Medvinsky, Edinburgh, unpublished; FIG. 3)

Genotyping eYFP (Clontech)

The enhanced Yellow Fluorescent Protein (eYFP) used to generate the bidirectional Aml1/Runx1-eYFP mice was obtained from pIRES-eYFP (ClonTech; PT3211-5)

| OLIGO | start | len | tm | gc % | any | 3′ | seq |
|---|---|---|---|---|---|---|---|
| eYFP-F | 68 | 20 | 60.04 | 50.00 | 4.00 | 3.00 | GACGTAAACGGCCACAAGTT(SEQ ID NO: 16) |
| eYFP-R | 406 | 20 | 59.81 | 55.00 | 5.00 | 2.00 | GTCCTCCTTGAAGTCGATGC(SEQ ID NO: 17) |

eYFP-R complementary sequence: GCATCGACTTCAAGGAGGAC(SEQ ID NO: 18)

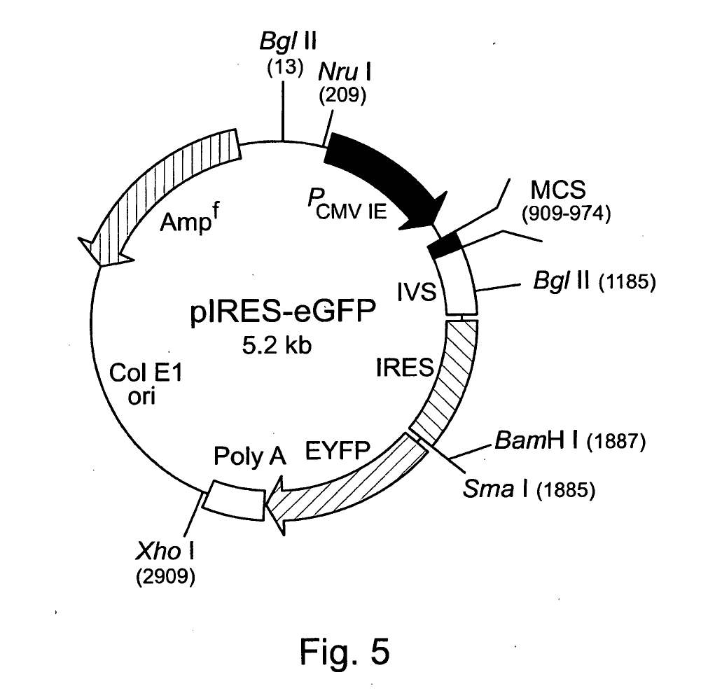

Product size: 339 bp
→both primers are located in eYFP
D. Rosa26-rtTA/HPRT-Aml1A/Runx1-IRES-eGFP mice (Edinburgh) (Liakhovitskaia A& Medvinsky A, Edinburgh, unpublished; FIG. 5)

### Genotyping Strategy

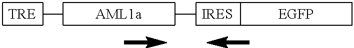

One primer is located in human Aml1A/Runx, the reverse primer binds to the IRES sequence.

| OLIGO-Name | start | len | tm | gc % | any | 3′ | seq |
|---|---|---|---|---|---|---|---|
| Aml1A/Runx1-F | 694 | 20 | 59.99 | 55.00 | 7.00 | 3.00 | AACCCTCAGCCTCAGAGTCA(SEQ ID NO: 19) |
| IRES-R | 993 | 20 | 59.99 | 50.00 | 7.00 | 2.00 | AGGAACTGCTTCCTTCACGA(SEQ ID NO: 20) |

Product size: 300

### Procedure

PCR reaction 20 μl:

1 μl DNA

2 μl 10×PCR reaction buffer

2 mM MgCl2

1 μl dNTPs (dATP, dCTP, dGTP, dTTP 2.5 mM each)

5% DMSO

0.5 μl primer1

0.5 μl primer2

0.5 μl primer3 (40 μmol/μl each)

1 U Taq polymerase

H2O up to 20 μl

### PCR Programs and Expected Band Sizes

A. Sox10-rtTA2^{s}-M2 mice (SEQ ID NO: 4)

3 min 94° C.

30 sec 94° C.

30 sec 60° C.

40 sec 72° C.->step 2-4 34x

5 min 72° C.

Expected size of band: 488 Kb and 618 Kb
B. Rosa26-rtTA mice (SEQ ID NO: 1)

3 min 94° C.

30 sec 94° C.

30 sec 50° C.

40 sec 72° C.->step 2-4 34x

5 min 72° C.

Expected size of band: 400 Kb
C. eYFP-TRE_{bl}Aml1A/Runx1 mice (SEQ ID NO: 2)

3 min 94° C.

30 sec 94° C.

30 sec 54° C.

40 sec 72° C.->step 2-4 34x

5 min 72° C.

Expected size of band: 170 Kb
D. Rosa26-rtTA/HPRT-Aml1A/Runx1-IRES-eGFP mice (SEQ ID NO: 3)

3 min 94° C.

30 sec 94° C.

30 sec 52° C.

40 sec 72° C.->step 2-4 34x

5 min 72° C.

Expected size of band: 231 Kb

### Analysis of PCR Products by Agarose Gel Electrophoresis

### Materials

DNA ladder 250 bp (Gi bco/Life Tech, cat#10596013)

Power Supply 300V (Owl Sep. Systems, cat#OSP300)

Gel combs, 50 tooth (Owl Sep. Systems, cat#D3-MT2D)

Gasketed UVT gel tray (Owl Sep. Systems, cat#D3-UVT-14)

Centipede wide format gel system (Owl Sep. Systems, cat#D3-14)

Casting chamber (Owl Sep. Systems, cat#D3-CST-14)

12.5 ul Matrix pipettor (Apogent Discoveries, cat#2019)

12.5 ul pipette tips from PCR protocol

P20 pipettor

Universal tips, 30 or 200 ul

### Reagents

50×TAE buffer [0.4 M Tris-base (96.88 g), 0.05M Sodium acetate (13.6 g sodium acetate-3 H2O), 0.01 M EDTA (7.44 g) in 2 liters water, pH to 7.6 with 12N HCl]
Agarose
Ethidium bromide (EtBr)

### Equipment

Gel electrophoresis

UV light box

### Preparations

1×TAE to make the gels and to fill the gel boxes. Each gel box requires at least one liter of fluid, and each gel requires 100 ml.
Mix 1 or 2 grams of agarose and 100 ml 1×TAE for each gel in a microwave (5-6 mins).
Cool the hot bottle and fit the gasketed gel tray into the casting chamber, making sure the gaskets are tight and the tray level.
Add 3-5 μl ethidium bromide to the cool agarose/TAE mixture, pour 100 ml into the tray.
Remove any air bubbles and fit four gel combs into the slots provided.
Let the gel set before using.

### Procedure

Remove plates from thermal cycler just prior to use.
Withdraw 4 ul (using tips saved from when you loaded the plasmid into the PCR) from each well of the PCR plate working one row at a time. Program the multichannel pipetter to fill with 2 ul and dispense 2 ul.
Load samples in gel.
Load 10 ul of DNA marker in each outside lane.
Run gels at 115 volts for 39 minutes.
Remove gel, read on UV light box and photograph.

### Breeding

The following combinations are used for breeding:

Sox10-rtTA2^{S}-M2 mice are mated with eYFP-TRE-Aml1A/Runx1 mice

Sox10-rtTA2^{S}-M2 mice are mated with HPRT-Aml1A/Runx1-IRES-eGFP mice

Both of these combinations yield the possibility to activate expression of Aml1A/Runx1 at the time when Sox10 is expressed in neurospheres or in stem cells in vitro and in vivo.
Sox10-rtTA2^{S}-M2 mice are mated with Rosa26-rtTA HPRT-Aml1A/Runx1-IRES-eGFP mice
   This combination yields the possibility to induce Aml1A/Runx1 expression in stem cells at the time of Sox10 expression and activate or deactivate expression Iof Aml1A/Runx1 later at the time of need. It also produces NCSCs with Sox10-rtTA and Rosa26-rtTA constructs. These NCSCs were used for subsequent transfection with Aml1 B/Runx1.
One heterozygote male is placed together with four heterozygote females in the evening
Females are checked for cervical plug in the morning the next day; presence of plug is defined as pregnancy Day 0.
   Culture of Boundary Cap Neural Crest Stem Cells (bNCSCs)

### Materials

Box with ice
Syringes and needles
Insect needle
Petri dishes
24-well plate (Nunc A/S, Catalog #142485 or equivalent)
15 ml tubes (Cellstar, Catalog #188271 or equivalent)
50 ml tubes (Cellstar, Catalog #227261 or equivalent)
Filter 0.2 μm (Schleicher & Schuell, Catalog #10462200 or equivalent)
Pipettes and pipette tips
Autoclaved instruments: Set 1: 2 pairs of curved scissors, 2 medium sized forceps and a small forceps. Set 2: a coarse forceps and a pair of scissors. Set 3: 2 coarse forceps, a fine forceps, a small knife and a pair of scissors for microdissection.

### Reagents

D-MEM/F-12 with L-glutamine and Hepes buffer (Invitrogen, cat #31330-038)
N-2 Supplement (1:100; Invitrogen, cat#17502-048)
B-27 (1:50; Invitrogen, cat#17504-044)
Serum albumin (0.1%, Sigma, cat#276855))
Basic fibroblast growth factor (bFGF, type 146aa; 20 ng/ml; R&D Systems, cat#233-FB-025)
Epidermal growth factor (EGF; 20 ng/ml; R&D Systems, cat#236-EG-01M)
Dimethyl sulfoxide (DMSO)—cryoprotection for freezing cells for storage in liquid nitrogen
70% ethanol
Phosphate buffered saline
Chloral hydrate (6%) for anaesthesia

### Equipment

Laminar flow hood

CO₂ incubator

Centrifuge 1-14 (Sigma, Labex instrument AB)

Dissection microscope

Inverted microscope

### Cell Dissociation Solutions

Stock Collagenase/Dispase 20 mg/ml (x20) in −20° C. freezer

Stock DNAses 10 mg/ml (x20) in −20° C. freezer

Prepare for DRG dissociation: 100 μl Coll/Disp+100 μl DNAse+1.8 ml N2 medium

### Culture Solutions

Solution 1: DMEM/F-12 (1×) liquid (Invitrogen), 1:1 with L-glutamine and Hepes buffer+N2 (volume 5 ml) (Invitrogen)+BSA 0.1%
Solution 2: DMEM/N2 medium+BSA 0.1% (Solution1)+B27 (1:50) (Invitrogen)
Solution 3: DMEM/N2medium+Collagenase/Dispase (Rosche)+DNase (Volume 500 μl) (Labkemi S)
DMEM/N2medium 450 μl+Collagenase/Dispase 25 μl+DNase 25 μl
Solution 4: Solution2 (DMEM/N2 medium+B27)+20 ng/ml EGF (RDS)+20 ng/ml bFGF (RDS)
Filter solutions.

### Procedure

The culture procedure is started in the evening of pregnancy Day 11, corresponding to embryonic stage E11.5
Place one large and three small Falcon tubes in a box with ice under the hood.
Place a glass-silicon Petri dish with thin needles for fixation of 11.5 days embryo under the hood. Sterilize with alcohol, rinse after 15-30 mins with sterile PBS, and thereafter fill it with N2 supplement and B27 medium.
Instruments to be used for embryo dissection: two straight thin sharp forceps; two curved) (90° tungsten needles, one holder for the needles, a small pair of scissors.
Three large Petri dishes are used for collection of embryos and for cleaning instruments.
Pipette with big blue tip. Heat up the blade and cut the blue tip. This pipette with blue tip is used for embryo transfer.
The uterus is removed from the anaesthetized pregnant mouse and placed in cold PBS.
Embryos are separated one by one using a pair of forceps and a small pair of scissors. Pick up the place between the embryos with a pair a forceps, cut along the back of the embryos, and they will fall out.
Embryos are taken with a pair of forceps around their heads, rinsed once in PBS, then in 70% ethanol, and again in PBS, and then immediately placed in a 15 ml tube with ca 3 ml of N2 medium.
One embryo at a time is picked up with blue pipette and placed in Petri dish for dissection (see item 1) filled with N2+B27 medium.
The tails from the embryo is collected in separate Eppendorfs and stored in −80° C. before DNA isolation.
Embryos are fixed on their back in the dissection Petri dish with needles—one at the head and the other between its legs. Remove all organs and other tissues down to the membrane covering the inside of the dorsal part of the embryo. Cut the membrane gently from up to down exactly in the middle with a small pair of scissors. When the membrane is cut all the way, the spinal cord can be seen (it is under it). Now remove with a pair of fine forceps connective tissue on each side keeping the forceps on both sides of the spinal cord at an angle of 45°. The dorsal root ganglia (DRGs) are now seen. Remove the skin behind the spinal cord and DRGs by holding the skin with forceps from the back on both sides from the back and pulling it from the cranial to the caudal end of the embryo. Now the spinal cord with all DRGs is isolated from most of the surrounding tissue. Remove the prospective vertebral processes and connective tissue found between and around the DRGs.
Collect the DRGs with tungsten needles, by keeping the spinal cord with a pair of forceps and separating 3-4 ganglia at a time. Take the DRGs with a 200 μl pipette and collect them separately from each embryo in a small Falcon tube on ice (some medium always goes with the DRGs when they are removed from the Petri dish and this amount is sufficient).
Let DRGs settle down before removing supernatant.
Add 500 μl Collagenase/Dispase (1 mg/ml) and DNase (0.5 mg/ml) into N₂ on ice.
Incubate ganglia for 20-30 minutes at 37° C. water bath. Shake the tubes once at halftime.
After incubation move the tubes carefully. Let ganglia settle and remove nearly all of the supernatant.
Rinse once in 500 μl N2 with B27 (1:50). Add this solution, let ganglia settle down, carefully remove supernatant.
Add 300 ul of fresh medium (the same as above; it should also have at least 0.1% BSA) and triturate carefully with fire polished Pasteur pipette with rubber tops. Be sure that no clusters of cells remain by checking the cell suspensions in a dissection microscope.
After dissociation, ˜100000-200000 cells/well were plated in a 24-well culture dish (this is the amount of cells obtainable from one embryo). Cells are placed directly into the wells, which are filled with 500 ul N2 medium+B27+EGF (20 ng/ml)+bFGF (20 ng/ml).
After 12 h non-adherent cells are removed together with half of the medium. Add up to 250 μl of fresh medium.
Medium is changed every 2^{nd} day (50% of the medium replaced with fresh medium) before neurospheres will be formed in around 3 weeks of culture.
After neurospheres were formed change medium every 3¹ day.
   Procedure for propagation of neurospheres
After 3 weeks the first neurospheres appear in propagation medium.
At this stage some attached cells can be still present in the well. In this case remove the neurospheres from the well and dissociate in new well with 400 μl of fresh medium by 1 ml pipette gently triturating for 15-16 times.
Separate dissociated cells in 2 wells and add 200 μl of fresh medium
The next day, after dissociation the cells may have to be separated, since the preceding dissociation induces fast growth of neurospheres—good to dissociate cells some days before planned transplantation.
Always keep medium around 37° C.
Check the cells every day and if some neurospheres grow faster and reach large size—transfer them to a new well and dissociate.
Avoid attached cells and keep the neurospheres transparent and light. Dark neurospheres have to be removed from the well.

### Transfection of Neurospheres

Neurospheres prepared from Sox10-rtTA2^{s}-M2 mouse embryos were transfected by electroporation with linearized DNA containing Aml1B/Runx1 (long Runx1 isoform).

### Materials

1 ml plastic pipettes

24 well culture dish

Eppendorf tubes

### Equipment

BioRad Electroporator at 320V, 200 μF

### Preparation of Plasmid

The plasmid eYFP-TRE_{bl}Aml1B/Runx1 was purified with Qiagen HiSpeed plasmid Midi kit (http://www.giagen.com). Protocol: pag 17-22 of HiSpeed plasmid Purification Handbook

### Procedure for Transfection

Two days before transfection part of neurospheres, harboring Sox10-rtTA were taken for RT-PCR analysis to confirm Sox10 expression.
The neurospheres, which harbored Sox10-rtTA construct and expressed Sox10 were transferred with 1 ml plastic pipette to separate wells of a 24-well dish in a concentration of 4−5×10⁶ cells per transfection sample.
5-10 μg of DNA was prepared for each sample (concentration 0.5 mg/ml).
Electroporation was performed in 4 mm gap cuvettes.
The neurospheres were carefully transferred first to an Eppendorf tube and all medium removed and then to 800 μl sterile PBS with 0.2% glucose.
The DNA was put into the cuvette, after which cells in PBS were added.
The following transfections were made: i) neurospheres from Sox10-rtTA embryos were transfected with the eYFP-TRE_{bl}Aml1B/Runx1 construct.
ii) neurospheres from [Sox10-rtTA+Rosa26-rtTA] embryos were transfected with the eYFP-TRE_{bl}Aml1B/Runx1 construct.
Electroporation was performed with BioRad Electroporator at 320 V, 200 μF.
After electroporation cells were left for 10 minutes in the cuvette and then put back into 24-well dish with pre-warmed propagation culture medium.
DOX treatment was started on the day after transfection, but not earlier than 24 hours after transfection.
Neurospheres were harvesting for RT-PCR analysis before and after transfection and DOX Treatment.
   Treatment with Doxycycline
Treatment with Doxycyclin (DOX; 1 μg/ml) was started 24 h after transfection.
24 hours after DOX treatment was started, monitoring of the expression of eYFP/eGFP was initiated, as an indicator of Aml1B/Runx1 and Aml1A/Runx1 induction respectively.
For monitoring eYFP/eGFP expression a fraction of neurospheres was taken from each well of the 24 wells-dish. When DOX induced eYFP/eGFP (and hence Aml1B/Runx1 or Aml1A/Runx1) expression was confirmed, the remaining untreated neurospheres were taken for differentiation assay with or without DOX treatment.

### Procedure for Preparation of Plasmid for Transfection

Use 500 ul DNA add 50 ul 3M NaAcetate
Shake (vortex) 1-2-min
Add 1250 ul 100% ethanol
Shake (vortex) 1-2 min
Place in freezer for 1 hour (−20° C.)
Centrifuge for 15 min at 14000 RPM
Remove supernatant and wash the pellet with 70% ethanol (500 ul)
Centrifuge for 5 min at 14000 RPM
Remove supernatant carefully and let the pellet dry at RT (around 15 min)
Add 250 ul H₂O
Shake (vortex) 2-3 min

### RT-PCR

Expression of different transcription factors in neurospheres during the period of their culture was analyzed. Since interest lies in expressing more than one transcription factor in a defined order, it is important to know beforehand if any of the transcription factors of interest are expressed in NCSCs during their period in culture, and, if so, if these expressions are stable or changes in a regular way concomitant with growth and dissociation of the neurospheres. For this purpose the following primers for the transcription factors indicated below were used.

### Primers

Sox10 (Acc. no. gi|149266994)
Sox10.F
(SEQ ID NO: 21)
TCTACACTGCCATCTCTGAC nt: 1691-1710
Sox10.R
(SEQ ID NO: 22)
CTCCTCCACTGCCAAGC nt: 1871-1887
Product length: 197 bp
Brn3a (Acc. no. NM_011143)
Brn3a-2-F
(SEQ ID NO: 23)
AGGCCTATTTTGCCGTACAACC nt 1384-1405
Brn3a-2-R
(SEQ ID NO: 24)
CTCCTCAGTAAGTGGCAGAGAATTTCAT nt 1536-1509
Product length: 152 bp
(Hjerling-Leffler et al, 2005)
Krox20 (Acc. no. NM_010118)
Krox20-forw
(SEQ ID NO: 25)
TCCTCCAGCGTCACCTC pos. 640-656
Krox20-rev
(SEQ ID NO: 26)
GGCGGCGATAAGAATGC pos. 818-802
Product length: 179 bp
Fkhl18/Fkh3/Foxs1 (Acc. no. NM_010226)
Foxs1-F
(SEQ ID NO: 27)
GGCCCTGTCAAGATAGACCA nt 451-470
Foxs1-R
(SEQ ID NO: 28)
ACTTTGCCGAGCACATCTCT nt 676-657
Product length: 226 bp
Neurogenin-1 (Acc. no. NM_010896)
Neurogenin1-forw
(SEQ ID NO: 29)
CCAGTAGTCCCTCGGCTTCAG pos. 848-868
Neurogenin1-rev
(SEQ ID NO: 30)
GTCGTGTGGAGCAGGTCTTTG pos. 941-921
Product length: 94 bp
Neurogenin-2 (Acc. no. NM_009718)
Neurogenin2-forw
(SEQ ID NO: 31)
CGGAGAAGCATCGTTATG pos. 1054-1071
Neurogenin2-rev
(SEQ ID NO: 32)
GGGAGAAGGATGGGAAG pos. 1161-1145
Product length: 108 bp
Aml1A/Runx1 (Acc. no. NM_009821 (murine Runx1))
Aml1A/Runx1-forw
(SEQ ID NO: 33)
CTCTGCTCCGTGCTACC pos. 1784-1800
Aml1A/Runx1-rev
(SEQ ID NO: 34)
GTCGTTGAATCTCGCTACC pos. 1972-1954
Product length: 189 bp
Additional primers for RT-PCR can be used for both
long and short isoforms):
hAml1A-forw
(SEQ ID NO: 35)
CCTCTGCTCCGTGCTGC pos. 1784-1800
hAml1A-rev
(SEQ ID NO: 36)
GGTCATTAAATCTTGCAACC pos. 1972-1954
Alternative primers for the Aml1A-IRES-EGFP
construct (only for Aml1/Runx1-eGFP mice):
Aml1A/Runx1-F
(SEQ ID NO: 37)
AACCCTCAGCCTCAGAGTCA
IRES-R
(SEQ ID NO: 38)
AGGAACTGCTTCCTTCACGA
The forward primer has 7 mismatches with the mouse
sequence (aaaccacaggatcagccaca), so it will not
compete for the primer. The IRES is not present in
the mouse genome, so the PCR fragment will be
specific for the transgene.
Runx3 (Acc. no. NM_019732)
Runx3-forw
(SEQ ID NO: 39)
TTCACGCTCACAATCAC pos. 954-970
Runx3-rev
(SEQ ID NO: 40)
CTGGTCTTCTATCTTCTGC pos. 1075-1058
Product length: 123 bp
Mts-1/S100A4 (Acc. no. NM_011311)
Mts1/S100A4-forw
(SEQ ID NO: 41)
ACAGCAACAGGGACAATGAAG pos. 240-260
Mts1/S100A4-rev
(SEQ ID NO: 42)
GCGAAGAAGCCAGAGTAAGG pos. 449-430
Product length: 210 bp
To monitor the up-regulation of Aml1A and Aml1B
expression in neurospheres the primers for IRES-
eGFP and eYFP (transfected long isoform),
respectively were used:
Aml1A/Runx1-F
(SEQ ID NO: 37)
AACCCTCAGCCTCAGAGTCA
IRES-R
(SEQ ID NO: 38)
AGGAACTGCTTCCTTCACGA
eYFP-F
(SEQ ID NO: 16)
GACGTAAACGGCCACAAGTT
eYFP-R
(SEQ ID NO: 17)
GTCCTCCTTGAAGTCGATGC

### Procedure

Total RNA is extracted from the neurospheres and transcribed into cDNA using random priming with MMLV reverse transcriptase.
For RT-PCR cDNA from 5 ng of total RNA is used for each reaction.
RT-PCR is conductec according to Lindblom et al (2006).
BioTools Taq polymerase is used according to the manufacturer's instructions.
SybrGreen is used as a detection reagent in 20 μl reactions.
Reactions are analyzed in a single color Biorad MylQ thermocycler.
Raw CT valuses are adjusted for variation in primer efficiency using LinReg PCR (Ramakers et al, 2003) and normalized for variation in cDNA concentrations using beta-tubulin.
All data are analyzed in Microscoft Excel.

### Immunohistochemistry

For immunohistochemistry whole neurospheres as well as dissociated cells, cultured in 24WD on polylysine coated coverslips, are used.
Culture medium is removed, cells washed with PBS and fixed in 0.15M phosophate buffered 4% formaldehyde (v/v), 14% saturated picric acid (v/v) for 10 minutes.
After fixation cells are washed carefully in cold PBS for 30 minutes and preincubation solution added directly to the wells with the coverslips for 1 hour.
Slides are incubated with primary antibodies (Table) at 8° C. overnight.
Slides are rinsed three times with PBS and incubated with appropriate secondary IgG antibodies (Jackson ImmunoResearch): Cy3 conjugated donkey anti-mouse (1:500, #715-165-150), FITC-conjugated donkey anti-rabbit (1:500, #711-095-152), Cy3-conjugated donkey anti-goat (1:500, #705-165-147), AMCA-conjugated donkey anti-rabbit (1:100; code#711-155-152), AMCA-conjugated donkey anti-mouse (1:100; #715-155-151) and Cy3-conjugated anti-guinea pig (1:500; #706-165-148) for 4 h at room temperature.
Some sections are incubated at 8° C. overnight with rhodamine conjugated Griffonia Simplicifolia Agglutinin isolectin B4 (1:100; Immunkemi, cat#FL-1201).
Slides are rinsed three times with PBS and coverslips removed from the wells with fine forceps.
Coverslips are mounted in 50% glycerol in PBS containing 100 mM propyl-gallate to prevent photobleaching.
Coverslips are examined in a Nikon Eclipse E800 microscope equipped with filters for separate or combined viewing of red, green and blue fluorescence.
For photography, a Nikon DXM1200F digital camera system was used. Aml1/Runx1 up-regulation is verified by the simultaneous expression of eYFP or eGFP (induced by the bidirectional promoter for Runx1 expression).

### Primary Antibodies

| Antibody/ | | | |
|---|---|---|---|
| lectin | Species | Source | Titer |
| Anti-beta- | Monoclonal mouse | Covance; cat#MMS-435P | 1:500 |
| tubulin | | | |
| Anti-beta- | Polyclonal rabbit | Covance; cat PRB-435P | 1:1000 |
| tubulin | | | |
| Anti-CGRP | Polyclonal rabbit | Chemicon; cat# AB5920 | 1:4000 |
| Anti-CNPase | Monoclonal mouse | Covance; cat# SMI-91R | 1:200 |
| Anti-GFAP | Polyclonal rabbit | DAKO; cat# Z0334 | 1:1000 |
| Anti-Ret | Polyclonal goat | R&D Systems; cat# af-482 | 1:20 |
| Anti-Sox10 | Polyclonal guinea | M. Wegner, Univ Erlangen | 1:1000 |
| | pig | | |
| RT-97 | Monoclonal mouse | Immunkemi; cat# VP-N752 | 1:500 |

### Animal Experiments

### Materials

1, 2 & 10 ml plastic syringes (Becton Dickinson)
Needles (Microlance, 21 G, 23G & 27G; Becton Dickinson)
Instruments: one large and one small pair of scissors, forceps, wound holder, bone cutter, clamps, eye scissor, jeweler's forceps
Cotton, spongostan
Pipette, black plastic box
Suture needle, silk, metal clips
Metal cannula for intracardiac perfusion, tubings
Microtome knives, specimen holders, microscope slides, coverslips, CO₂ for specimen freezing

### Reagents

Chloral hydrate (Apoteket, Sweden)
Sterile water
Cyclosporine (Sandimmun®, Novartis; 50 mlg/ml)
Doxycycline (Doxyferm®, Nordic Drugs; 20 mg/ml)
Doxycycline hyclate (Sigma Aldrich, cat# D9891-25G)
NaCl, phosphate buffered saline
Formaldehyde (38%), picric acid (saturated in distilled water)
Glycerol, paraphenylene diamine

### Equipment

Operating microscope
Perfusion pump (Masterfiex, Cole-Parmer, model 7553-75, pumphead 6-600 RPM)
Cryostat (Microm)

### Preparation of Anaesthetics

0.5 ml of Xylazine (Rompun, 20 mg/ml), 2 ml of Ketamine (50 mg/ml)+7.5 ml water for injection.

### Preparation of Perfusion Solution and Tissue Storage Solution

Physiological saline (body temperature) for rinsing the vascular system before fixative.
Fixative consisting of 4% formaldehyde (v/v), 14% saturated picric acid (v/v) in 0.15 M phosphate buffered, pH 7.35-7.45.
For cryoprotection: 15% sucrose in 0.15M phosphate buffer, pH 7.35-7.45.

### Transplantation Procedure

Animals are anaesthetized by intraperitoneal injection placed with their ventral part down. All operations are made under an operating microscope.
Lumbar dorsal root ganglia L4 and L5 are exposed unilaterally via a partial laminectomy.
Dorsal roots L4 and L5 are cut immediately proximal to the corresponding ganglia.
The L4 and L5 dorsal root ganglia are carefully removed.
The resulting cavities are gently filled with neurospheres from either TRE-Runx1 transfected Sox10-rtTA mouse embryos or from [Sox10-rtTA x TRE-Runx1]F1 embryos.
To facilitate handling and visualization of the neurospheres prior to transplantation, they are placed in a black plastic box filled with a small amount of culture medium.

### Postoperative Treatment

Animals are given daily intraperitoneal injections of Cyclosporine (350 mg/100 g b.wt).
Mice are given DOX in their drinking water (0.3 g doxycycline hyclate, 5 g sucrose in 100 ml water), or just drinking water.

### Preparation of Perfusion Solutions and Termination of Experiment

One week to two months after transplantation, the animals are re-anesthetized, and placed with their back down on a lab-bench with outflow system.
The chest is opened, the internal thoracic arteries clamped, and the heart exposed and held with a pair of forceps.
The left ventricle is punctured with a 21 G needle connected to the tubing from the perfusion pump.
To minimize the risk of damaging blood vessels emerging from the heart, a rubber stop is fixed to the needle just behind its opening.
About 20 ml of physiological saline is perfused, followed by ca 75 ml of fixative.

### Tissue Preparation

After perfusion, the transplant sites, corresponding dorsal roots and spinal cord segments are carefully dissected.
Place tissue in cold fixative for ca four hours.
Place tissue overnight in cold phosphate buffered saline containing 15% sucrose for cryoprotection.
Section tissue blocks the following day on a cryostat (thickness 8 um on a cryostat)
Placed sections on microscope slides
Store slides at −20° C. for later immunohistochemical processing (see section on immunohistochemstry).

### Microscopic Analysis

Slides are viewed and analysed in a Nikon Eclipse E800 fluorescence microscope equipped with filters for separate or combined viewing of red, green and blue fluorescence.
For photography, a Nikon DXM1200F digital camera system was used. Transplanted cells are identified by their expression of eYFP or eGFP (induced by the bidirectional promoter for Runx1 expression) in combination with neuronal markers.

### Results

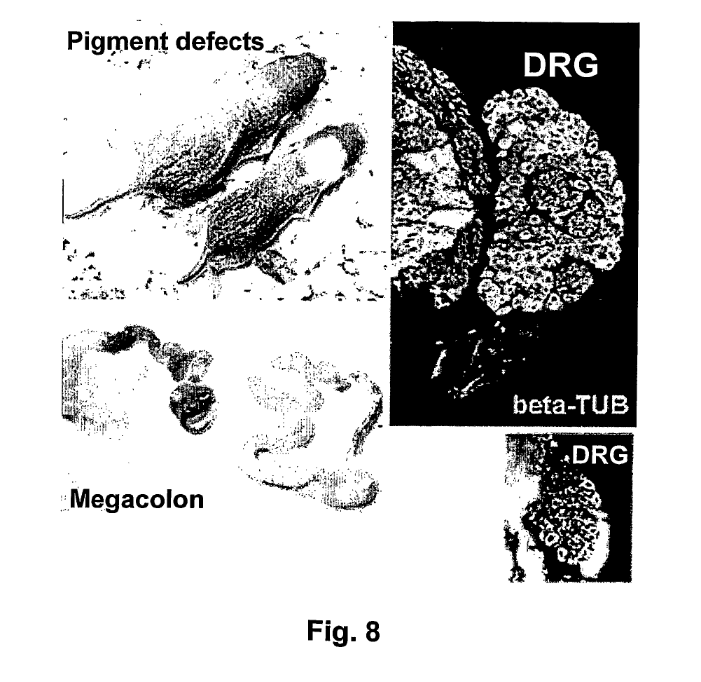

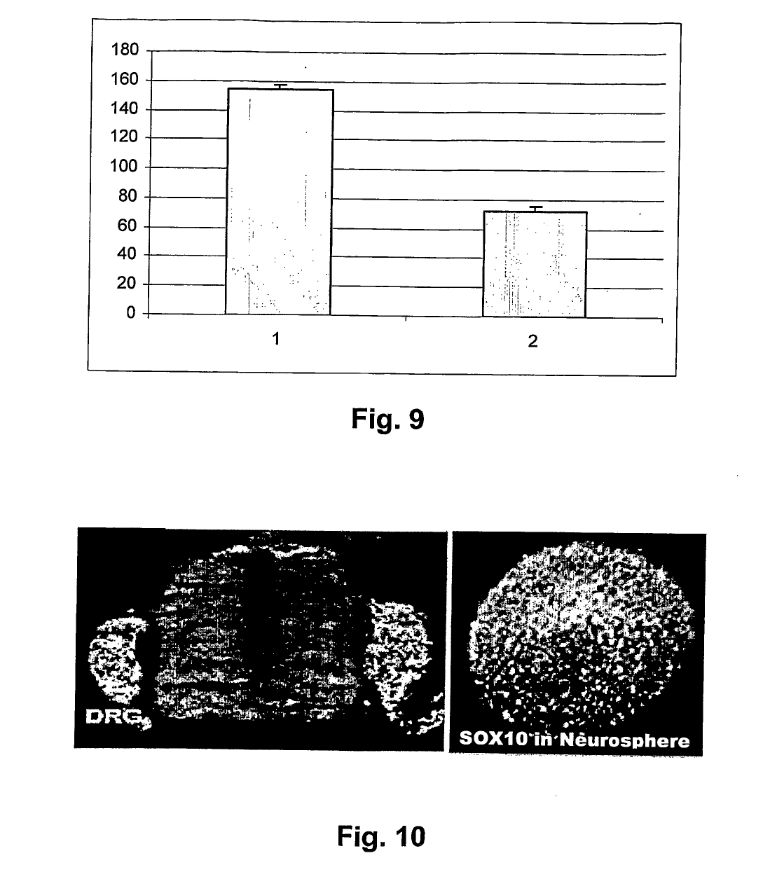

The functionality of the Tet-system in vivo was first confirmed. Sox10-rtTA female mice were bred with TRE-Aml1A/Runx1 males and DOX was given in the drinking water to the pregnant females pregnancy days 7 to 8 and 11.5 to 14, which correspond to the period NCSC migration. As a result of ectopic Aml1A/Runx1 expression during NCSC migration the offsprings of these females were smaller than normal and displayed pigment defects, megacolon, and severe reduction in size of DRGs (FIG. 8). The number of DRG neurons was reduced by about 50% (FIG. 9).

### Expression of Sox10 In Situ and In Vitro

Thereafter, it was confirmed by way of immunohistochemistry that Sox10 is expressed in E11 DRGs in the transgenic mice (FIG. 10 left), which are in accordance with invention used for stem cell preparation.

Moreover, expression of Sox10 in NCSC neurospheres (FIG. 10 right) was confirmed with immunohistochemistry during two weeks after neurospheres were formed and until 5 weeks in culture.

Subsequently, the expression of different transcription factors including Sox10 at different time points in NCSC neurospheres was analyzed with RT-PCR.

### RT-PCR Results

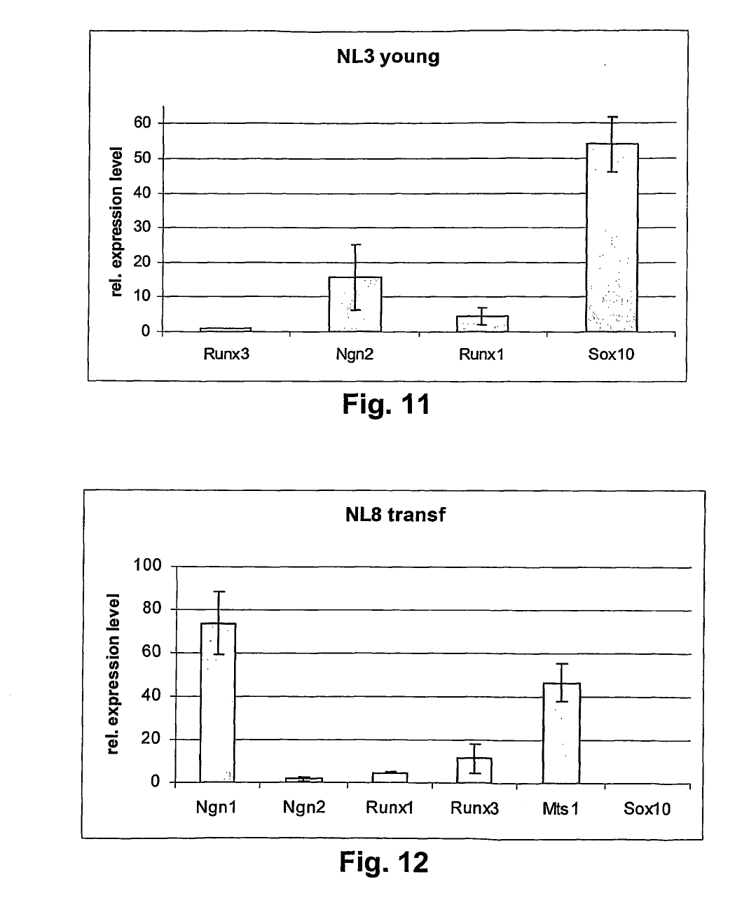

Sox10 is expressed transiently for only two weeks after neurospheres have developed (up to five weeks in culture, two passages; FIG. 11), giving a sufficient time for activating the Tet-regulatory system in cultured or transplanted NCSCs.

Three weeks old neurospheres from Sox10-rtTA embryos were transfected with Aml1B/Runx1 long isoform and 24 hours later DOX was applied. Twenty-four hours later the cells were harvested for RT-PCR. It is confirmed that transfection switched the expression of transcription factors towards differentiation of nociceptor neurons, which in vivo develop from an Ngn1 expressing cohort (FIG. 12).

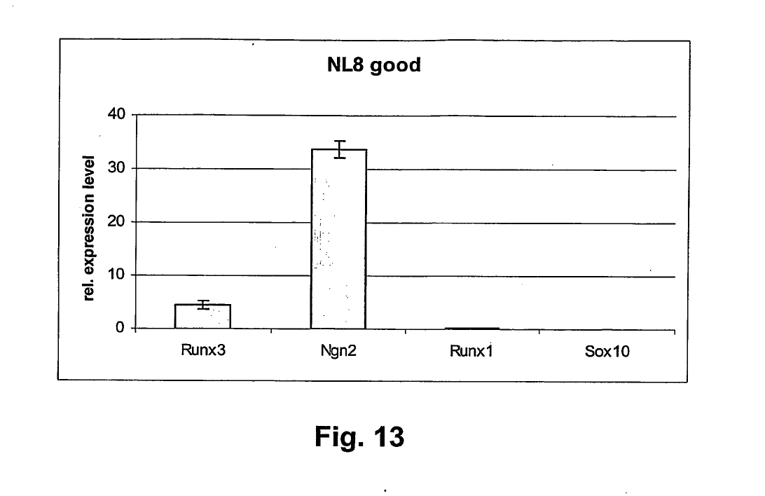

Neurospheres, which were not subjected to DOX mimicked the normal transcription factor expression pattern and expressed Ngn2 (FIG. 13), but not Ngn1.

Conclusion: Transfection and subsequent DOX application induced in the NCSCs the expression of Ngn1 and Runx1, which are specific transcription factors for nociceptor sensory neuron differentiation during DRG development in vivo. Thus, modulation with the Tet-system in vitro of the expression of the transcription factors critical for this process was enabled.

Next it was tested in vitro and in vivo if the activation of these transcription factors is sufficient for specific DRG differentiation.

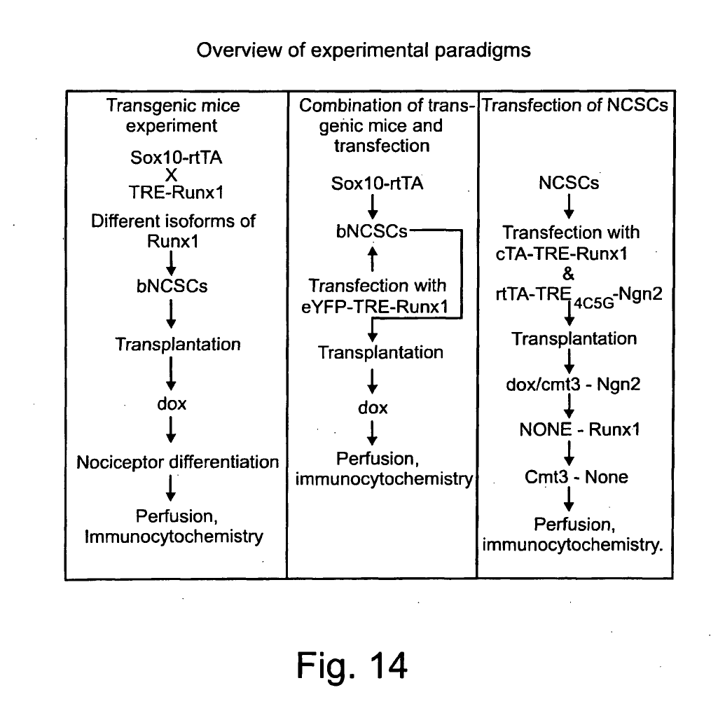

For this purpose three different paradigms are used, which are presented like a summary of the different protocols used for Tet-system mediated activation of Runx1 or Ngn2/Runx1 expression (FIG. 14)

### In Vitro Experiments

Sox10-rtTA2^{s}-M2 with eYFP-TRE-Aml1A/Runx1 mice were bred (fist column in the overview scheme) and neurospheres prepared from E11 embryos. The individual embryos were genotyped and DRGs from separate embryos cultured in separate wells. The genotyping of embryos showed which of the embryos were harboring both constructs of Tet-regulatory system. Only the neurospheres with Sox10-rtTA2^{s}-M2 and eYFP-TRE-Aml1A/Runx1 were taken for the experiments. After neurospheres were formed and expression of Sox10 was confirmed with RT-PCR the cells were divided in two different groups and mitogens removed from the culture medium. To one of the groups DOX was added to the culture medium during six days (one change in three days) and to the other group no DOX was added.

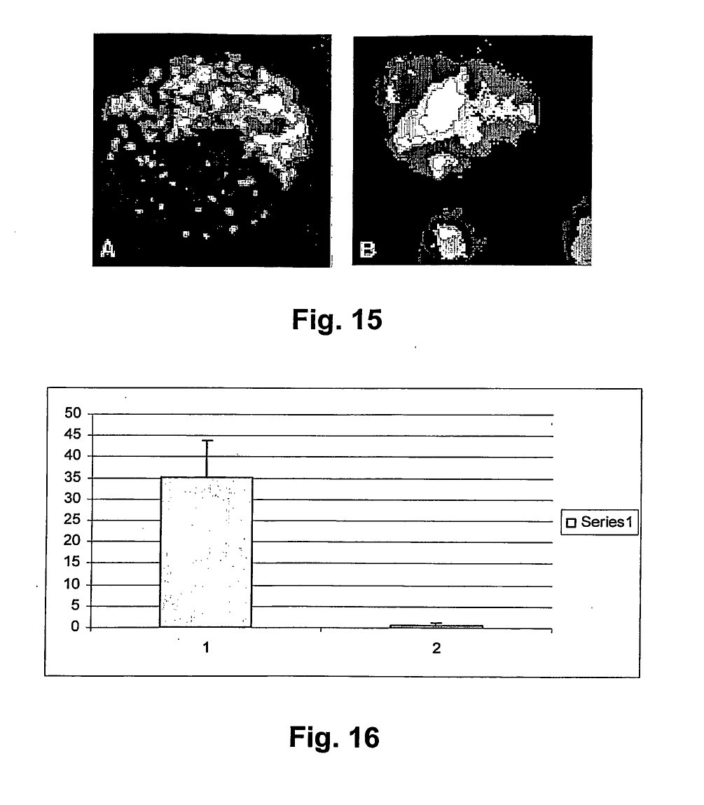

The expression of eYFP in response to DOX treatment was confirmed (FIG. 15 left) 24 hours after treatment was started. DOX activated neurospheres were transferred to a 24 well dish with D-poly-lysine coated coverslips for differentiation assay. Alternatively, cells from dissociated neuropheres were transferred to a 96 well dish and DOX treatment continued. Untreated neurospheres were used as control.

In a second group of experiments neurospheres from E11 embryos from Sox10-rtTA2^{s}-M2 mice were used (second column in the overview scheme). The genotyping of embryos gave information as to which of the embryos harbored the Sox10-rtTA2^{s}-M2 construct. Only these neurospheres were used for the experiment. After neurospheres had formed and Sox10 expression confirmed with RT-PCR they were transfected with linearized Aml1B/Runx1 isoform. After transfection the cells were allowed to recover for one day and DOX was applied on the next day. Twenty-four hours later eYFP expression was confirmed (FIG. 15 right).

DOX activated neurospheres were transferred to a 24 well dish with D-poly-lysine coated coverslips for differentiation assay. Alternatively, cells from dissociated neuropheres were transferred to a 96 well dish and DOX treatment continued. Untreated neurospheres were used as control.

Already on the second day after transfection with Aml1B/Runx1 isoform DRG neurons were observed, having a characteristic morphology on the surface of neurospheres. At this stage, no DRG neurons with these characteristics were seen in control cultures, as well as in cultures of neurospheres harbouring Aml1A/Runx1 isoform and treated with DOX.

The number of cells with typical DRG shape was calculated on six coverslips from each experiment (Neurospheres transfected with Aml1B/Runx1 and Neurospheres harbouring Aml1A/Runx1) and found that DOX induces the DRG differentiation in culture (FIG. 16).

After five days in culture cells from both DOX-treated groups were fixed in 4% paraformaldehyde, and immunostained for beta-tubulin, glial fibrillary acidic protein (GFAP), calcitonin gene-related peptide (CGRP), heavy neurofilament protein (antibody RT-97) and glial DRG markers Mts1/S100A4, Sox10 and labelled for Griffonia Simplicifolia Agglutinin isolectin B4 binding. The coverslips were then placed on slides and examined in a Nikon Eclipse fluorescence microscope.

Part of the DOX treated neurospheres was taken for RT-PCR to confirm the expression of Aml1B/Runx1 and Aml1A/Runx1.

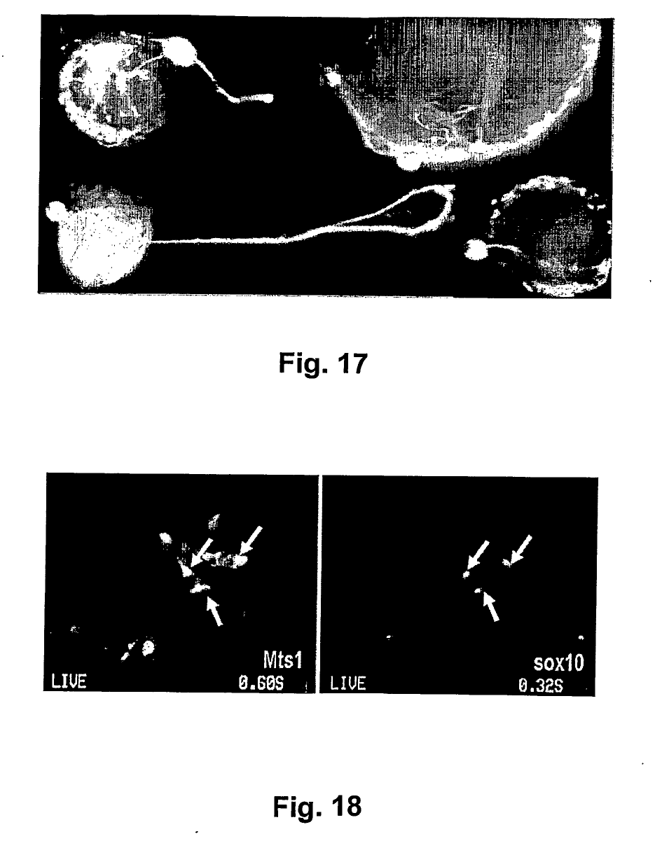

The data show that cultures transfected with the long Aml1B/Runx1 isoform and treated with DOX developed to DRG neurons with distinct DRG shape (FIG. 17).

The cultures harboring short Aml1A/Runx1 isoform during this period of time did not differentiated to DRG neurons and some of the cells expressed peripheral glial markers Sox10 and Mts1/S100A4 (FIG. 18).

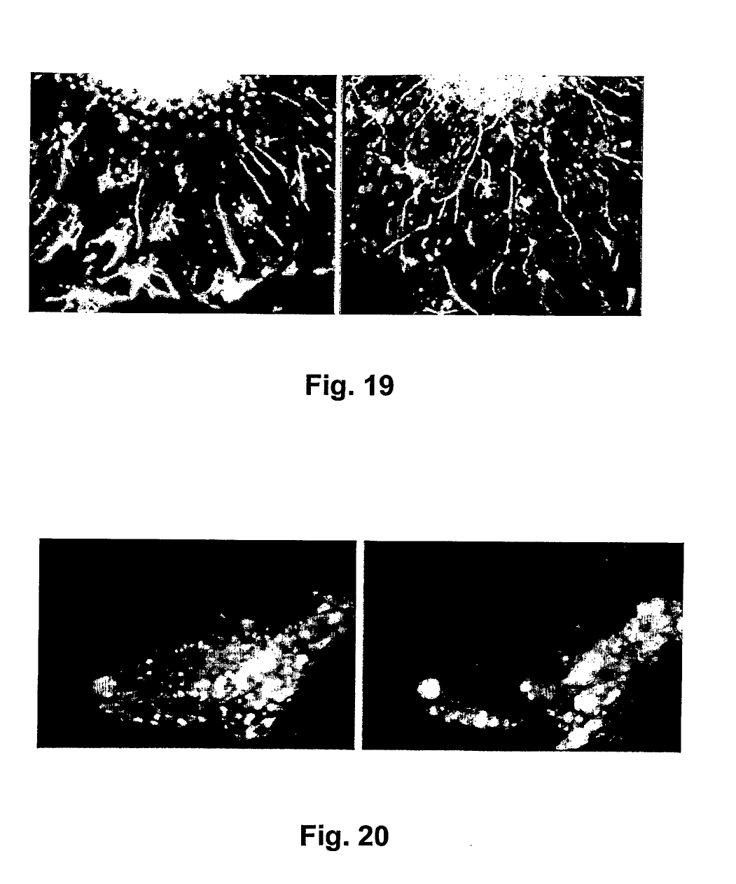

These experiment demonstrated that if Aml1B/Runx1 is conditionaly up-regulated in NCSCs, they differentiate to DRG neurons in vitro (FIG. 19 right). If the short Aml1A/Runx1 isoform is up-regulated, the differentiation of DRG neurons is prevented and NCSCs preferably differentiate to glial cells (FIG. 19 left).

### In Vivo Experiments

The neurospheres which were formed from Sox10-rtTA2^{s}-M2 embryos and were transfected with Aml1B/Runx1 were cultured for two hours in the presence of Hoechst before transplantation The L4 and L5 DRGs from adult C3H non transgenic mice were removed and collected neurospheres were placed into the DRG cavity. Treatment with DOX was started directly after transplantation and continued until mice recipients were sacrificed. During this period the mice received daily cyclosporine A treatment. One week after transplantation the mice were perfused with fixative and material was frozen and cut on a cryostat. The sections were stained with markers for DRG neurons and glia.

The 3-weeks transplant were identified by the presence of Hoechst (FIG. 20 left) in the transplanted cells as well as by their eYFP expression (FIG. 20 right), which was induced by DOX administration, thereby confirming the Aml1B/Runx1 or Aml1A/Runx1 expression in the neurospheres after transplantation.

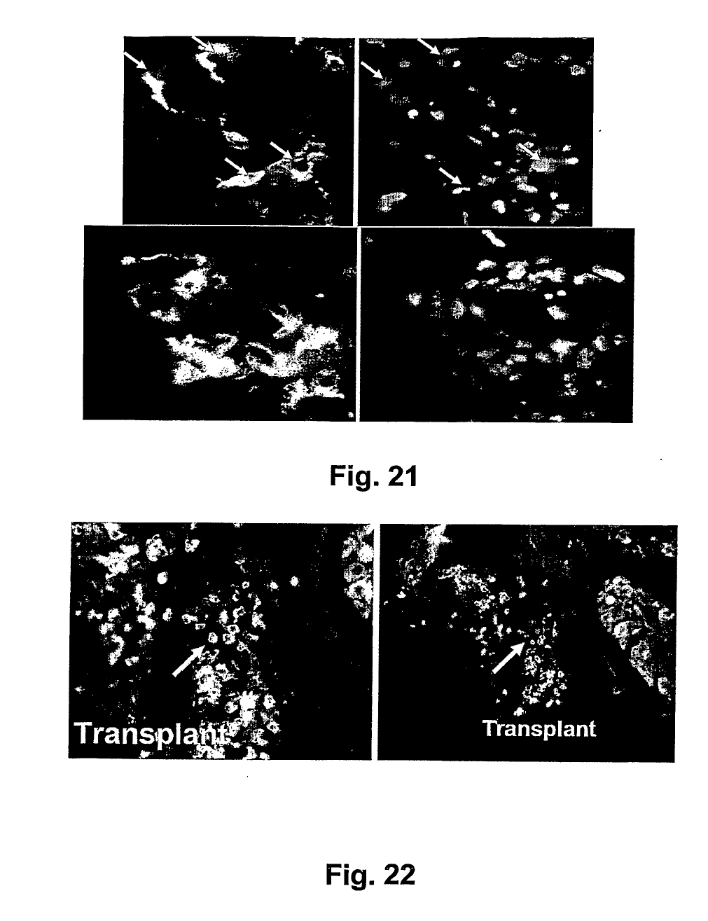

Around 10% of transplanted Hoechst positive cells (FIG. 21 right) in 3 weeks after transplantation expressed the neuronal marker beta-tubulin (bTUB) (FIG. 21 left).

Alternatively the transplanted cells were identified by their location and morphological features—the developing stem cells were located in the DRG cavity, expressed specific neuronal marker bTUB and the size of the cells was smaller compare to the intact DRG neurons (FIG. 22).

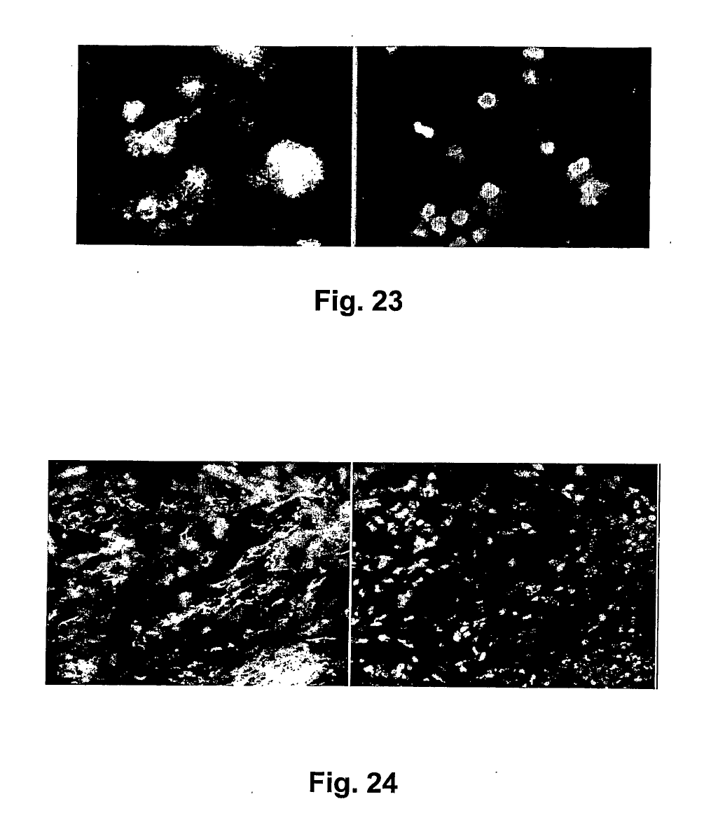

When recipients with Sox10-rtTA transplanted neurospheres transfected with Aml1B/Runx1 isoform, received DOX after transplantation, it was observed that transplanted cells bind rhodamine conjugated isolectin B4 (FIG. 23). When transplanted cells contained sox10-rtTA and Rosa26-rtTA and had been transfected with long Aml1B/Runx1 isoform, robust binding of B4 in transplanted cells (FIG. 24 left) was observed. The same cells contained, Hoechst which was applied to the cells before transplantation (FIG. 24 right).

The neurospheres from DRGs of embryos harboring short Aml1A/Runx1 isoform also expressed neuronal marker beta-tubulin.

However, binding of B4 in the transplanted cells was not observed.

The results show i) that neural crest stem cells, containing Tet system, after transplantation can be induced to express gene(s) of interest which is located under tetracycline inducible transactivator control. ii) that the ectopic expression of specific transcription factor for nociceptor neuron diversification Aml1B/Runx1 guides the differentiation of NCSCs towards a neurochemical phenotype tipical of nociceptor sensory neurons, iii) that using exogenous drug-regulatable systems it is possible to guide differentiation of stem cells after transplantation. These results prove the principle that activation of key transcription factors can guide the differentiation of immature stem cells toward desired type of neurons.

### Independent Tet-Regulation of Two Transcription Factors for Human Stem Cell Differentiation

Thus, the proof of princiople is demonstrated, i.e. that the combination of critical transcription factors can guide the differentiation of stem cells from transgenic mice towards the desired type of neurons after transplantation.

However, in human transplants it is not possible to combine genetically inserted Tet-systems or any other transgenic regulatory systems. The necessary components have to be delivered to the human stem cells and then after transplantation be activated from outside.

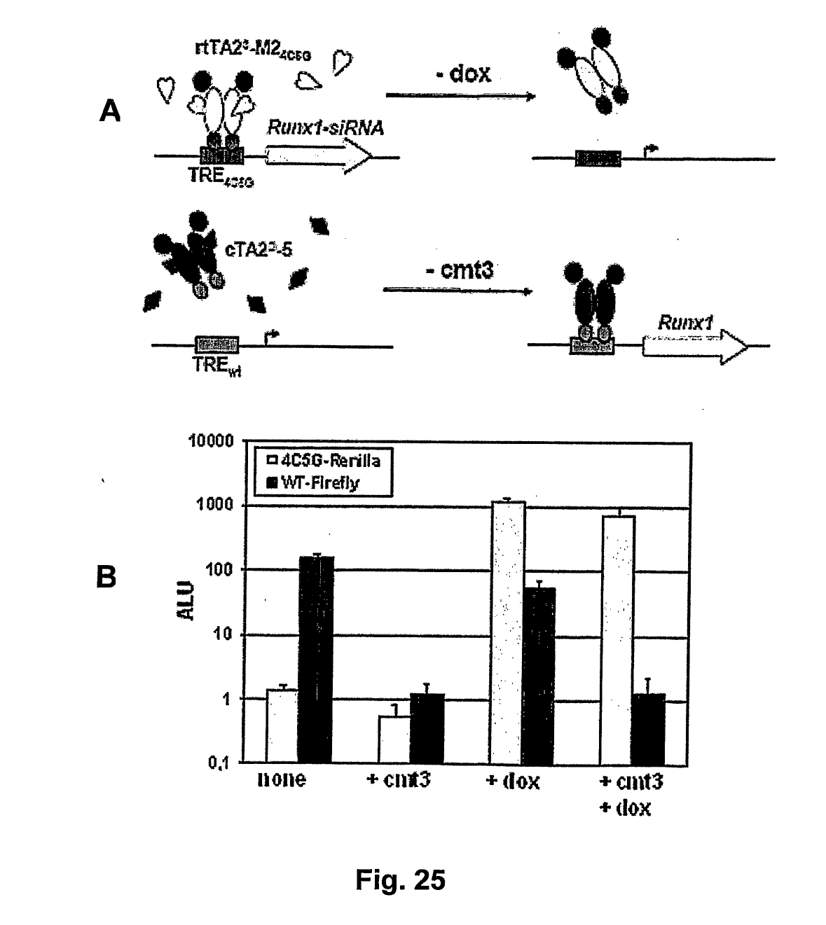

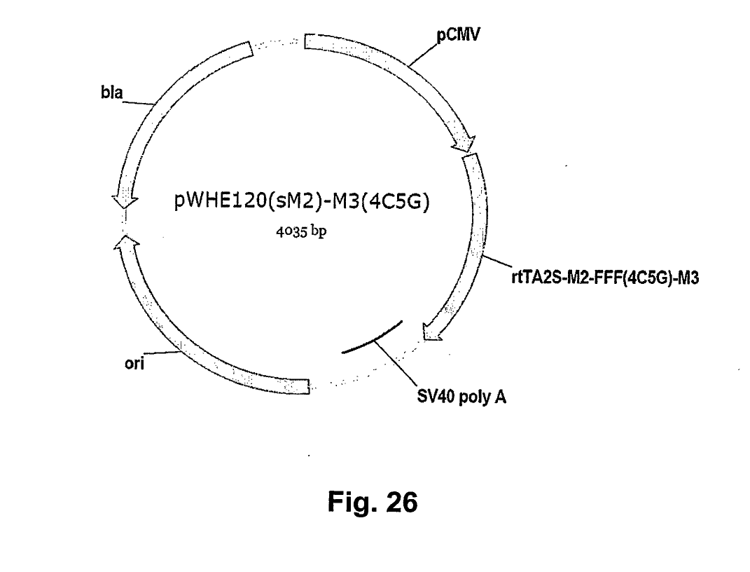

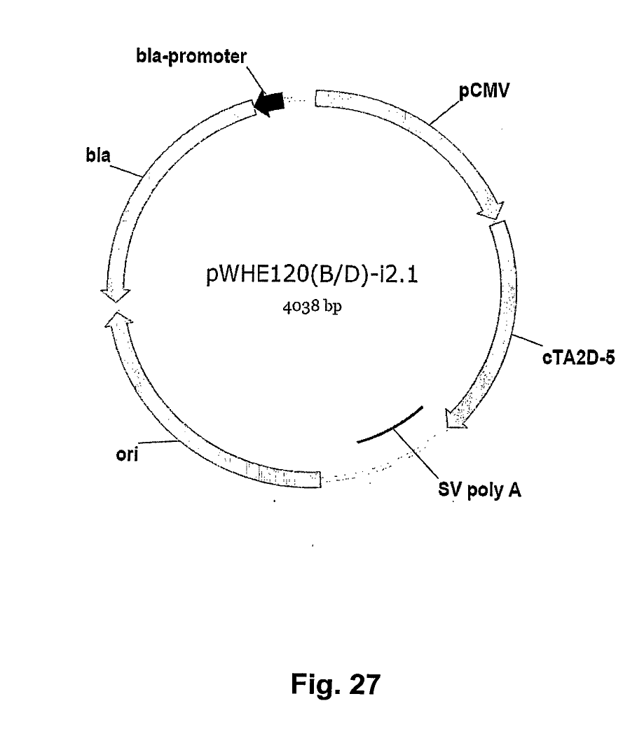

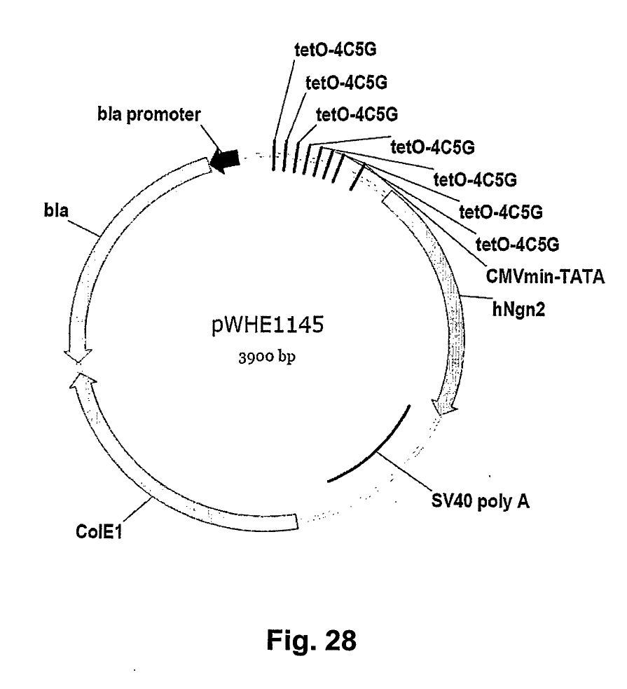

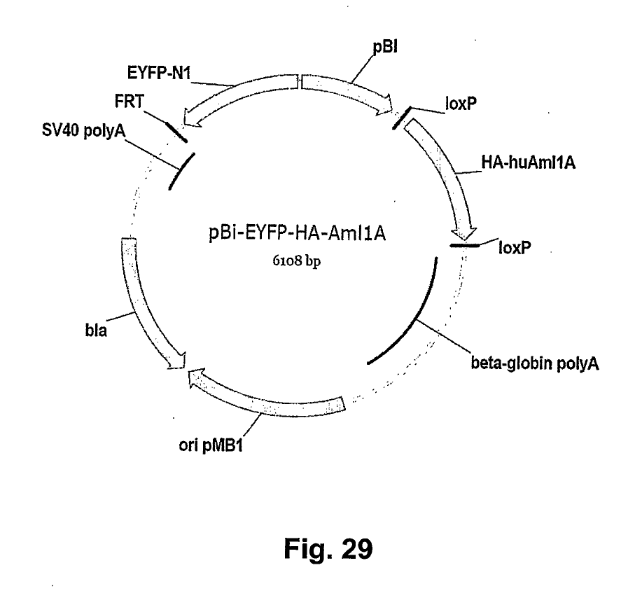

Recently two transcription factors have been constructed—cTA2^{D}-5 and rtTA2^{s}-M2_{4C5G}—with exclusive specificities for DNA binding (wt TRE/4C5G TRE), effector recognition (cmt3/dox) and homodimer formation (class D/class B) (Dr. Christian Berens, Erlangen University). All these components were tested for the first time in eukaryotic cells to determine their regulatory properties (C. Berens, unpublished). The Hela cell line HLRF33/1141, which contains reporter genes for firefly luciferase under control of a wt TRE element and Renilla luciferase under control of a 4C5G-specific TRE element was transiently transfected with plasmids encoding the transactivators and incubated with different combinations of effectors. The results are shown in FIG. 25.

Expression of Renilla luciferase controlled by rtTA2^{s}-M2_{4C5G} (FIG. 26) (SEQ ID NO: 7) is increased about 1000-fold and only in the presence of (FIG. 25 light columns 3 and 4). Addition of cmt3 leads to a ˜100-fold decrease in firefly luciferase activity, controlled by cTA2^{D}-5 (FIG. 27 (SEQ ID NO: 8)) (FIG. 25 dark columns 2 and 4), while expression of firefly luciferase is only slightly, less than 2-fold, reduced in the presence of dox (FIG. 25).

These findings show that it is possible to achieve exclusive expression of either firefly luciferase in the absence of effectors and of Renilla luciferase in the presence of both cmt3 and dox. The regulatory system is thus functional and can be applied for target gene expression in stem cells.

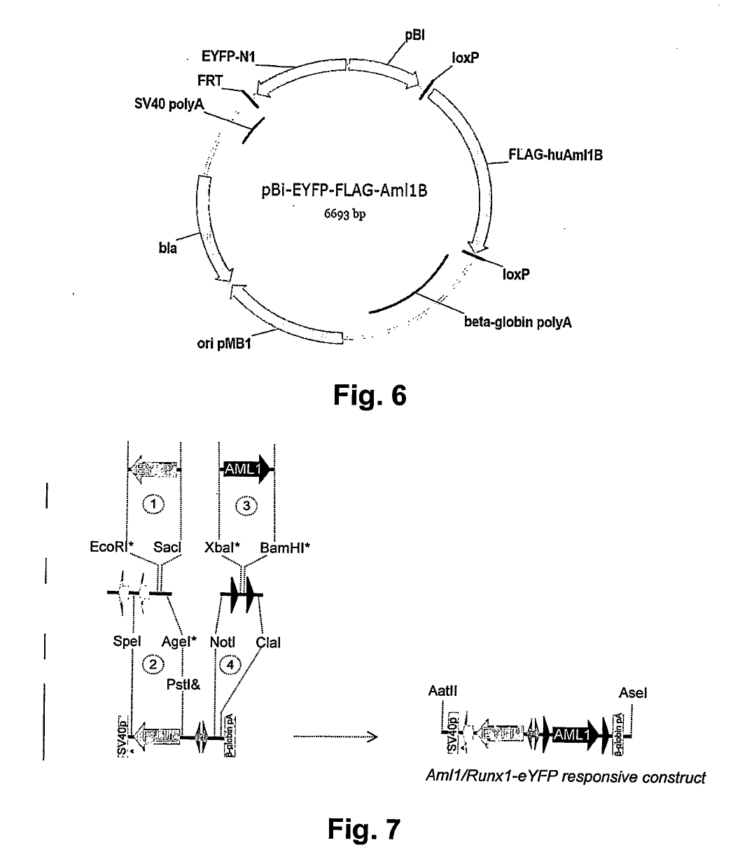

This system is now adapted for independent conditional activation of Ngn2 (FIG. 28 (SEQ ID NO: 9) and Aml1B/Runx1 (FIG. 6) and Aml1A/Runx1 (FIG. 29) genes corresponding to Renilla luciferase and firefly luciferase, respectively in the experiment described above.

To activate 3 genes of interest, which are crucial for guiding differentiation of stem cells to desired type of cells after transplantation, the artificial regulatory network interconnecting the tetracycline (TET-), streptogramin-(PIP-) and macrolide (E-) systems may be applied (Kramer et al, 2003).

Even though the invention has been described particularly with respect to the examples and figures, it should be understood that these are only made to illustrate the essential features of the invention, and that deviations from the disclosed details may naturally be made by a man skilled in the art while still being comprised by the invention as claimed.

### REFERENCES

Ackermann A M, Gannon M (2007) Molecular regulation of pancreatic beta-cell mass development, maintenance, and expansion. J Mol Endocrinol 38:193-206.
Andersson E, Jensen J B, Parmar M, Guillemot F, Bjorklund A (2006) Development of the mesencephalic dopaminergic neuron system is compromised in the absence of neurogenin 2. Development 133:507-516.
Andersson E K, Irvin D K, Ahlsio J, Parmar M (2007) Ngn2 and Nurr1 act in synergy to induce midbrain dopaminergic neurons from expanded neural stem and progenitor cells. Exp Cell Res 313:1172-1180.
Babetto E, Mangolini A, Rizzardini M, Lupi M, Conforti L, Rusmini P, Poletti A, Cantoni L (2005) Tetracycline-regulated gene expression in the NSC-34-tTA cell line for investigation of motor neuron diseases. Brain Res Molec Brain Res 140:63-72.
Baharvand H, Jafary H, Massumi M, Ashtiani S K (2006) Generation of insulin-secreting cells from human embryonic stem cells. Dev Growth Differ, Dev Growth Differ 48:323-332
Beech R D, Cleary M A, Treloar H B, Eisch A J, Harrist A V, Zhong W, Greer C A, Duman R S, Picciotto M R. Nestin promoter/enhancer directs transgene expression to precursors of adult generated periglomerular neurons. J Comp Neurol. 2004 Jul. 12; 475(1):128-41.
Bellion A, Métin C (2005) Early regionalisation of the neocortex and the medial ganglionic eminence. Brain Res Bull 66:402-409.
Berendse H W, Ponsen M M (2006) Detection of preclinical Parkinson's disease along the olfactory trac(t). J Neural Transm Suppl. (70):321-325.
Berens C, Hillen W (2003) Gene regulation by tetracyclines. Constraints of resistance regulation in bacteria shape TetR for application in eukaryotes. Eur J Biochem 270:3109-3121.
Bliss T, Guzman R, Daadi M, Steinberg G K (2007) Cell transplantation therapy for stroke. Stroke 38(2 Suppl):817-826.
Bondurand N, Natarajan D, Barlow A, Thapar N, Pachnis V (2006) Maintenance of mammalian enteric nervous system progenitors by SOX10 and endothelin 3 signalling. Development 133:2075-2086.
Brännvall K, Sandelin M, Wallenquist U, Forsberg-Nilsson K, Aldskogius H, Kozlova E N (2006) Central nervous system stem/progenitor cells form neurons and peripheral glia after transplantation to the dorsal root ganglion. Neuroreport 17:623-628.
Buckingham M, Relaix F (2006) The Role of Pax Genes in the Development of Tissues and Organs: Pax3 and Pax7 Regulate Muscle Progenitor Cell Functions. Annu Rev Cell Dev Biol Sep 8; [Epub ahead of print]
Castillo C G, Mendoza S, Freed W J, Giordano M (2006) Intranigral transplants of immortalized GABAergic cells decrease the expression of kainic acid-induced seizures in the rat. Behav Brain Res 171:109-115.
Chen C L, Broom D C, Liu Y, de Nooij J C, Li Z, Cen C, Samad O A, Jessell T M, Woolf C J, Ma Q (2006) Runx1 determines nociceptive sensory neuron phenotype and is required for thermal and neuropathic pain. (2006) Neuron 49:365-377.
Choong C, Rao M S (2007) Human embryonic stem cells. Neurosurg Clin N Am 18:1-14.
Cobos I, Calcagnotto M E, Vilaythong A J, Thwin M T, Noebels J L, Baraban S C, John L Rubenstein J L R (2005) Mice lacking Dlx1 show subtype-specific loss of interneurons, reduced inhibition and epilepsy. Nat Neurosci 8:1059-1068.
Corbel S Y, Rossi F M (2002) Latest developments and in vivo use of the Tet system: ex vivo and in vivo delivery of tetracycline-regulated genes. Curr Opin Biotechnol 13:448-452.
Diamond I, Owolabi T, Marco M, Lam C, Glick A (2000) Conditional gene expression in the epidermis of transgenic mice using the tetracycline-regulated transactivators tTA and rTA linked to the keratin 5 promoter. J Invest Dermatol 115:788-794.
Dobkin B H (2007) Behavioral, temporal, and spatial targets for cellular transplants as adjuncts to rehabilitation for stroke. Stroke 38(2 Suppl):832-839.
Engel T, Hernandez F, Avila J, Lucas J J (2006) Full reversal of Alzheimer's disease-like phenotype in a mouse model with conditional overexpression of glycogen synthase kinase-3. J Neurosci. 26:5083-5090.
Fritzsch B, Beisel K W, Hansen L A (2006) The molecular basis of neurosensory cell formation in ear development: a blueprint for hair cell and sensory neuron regeneration? BioEssays 28:1181-1193.
Fukuda K, Fujita J. Mesenchymal, but not hematopoietic, stem cells can be mobilized and differentiate into cardiomyocytes after myocardial infarction in mice. Kidney Int. 2005 November; 68(5):1940-1943.
Gangaram-Panday S T, Faas M M, de Vos P (2007) Towards stem-cell therapy in the endocrine pancreas. Trends Mol Med 13:164-173.
Gopalkrishnan R V, Christiansen K A, Goldstein N I, DePinho R A, Fisher P B (1999) Use of the human EF-1alpha promoter for expression can significantly increase success in establishing stable cell lines with consistent expression: a study using the tetracycline-inducible system in human cancer cells. Nucleic Acids Res 27:4775-4782.
Gossen M, Bujard H (1992) Tight control of gene expression in mammalian cells by tetracycline-responsive promoters. PNAS 89:5547-5551.
Gossen M, Bujard H (2002) Studying gene function in eukaryotes by conditional gene inactivation. Annu Rev Genet. 36:153-173.
Gossen M, Freundlieb S, Bender G, Muller G, Hillen W, Bujard H (1995) Transcriptional activation by tetracyclines in mammalian cells. Science 268:1766-1769.
Goverdhana S, Puntel M, Fux C et at (2005) Regulatable gene expression systems for gene therapy applications: progress and future challenges. Mol Ther 12:189-211.
Grill M A, Bales M A, Fought A N, Rosburg K C, Munger S J, Antin P B (2003) Tetracycline-inducible system for regulation of skeletal muscle-specific gene expression in transgenic mice. Transgenic Res 12:33-43.
Guillemot F (2007) Cell fate specification in the mammalian telencephalon. Prog Neurobiol Mar 7; [Epub ahead of print]
Harada T, Harada C, Parada L F (2007) Molecular regulation of visual system development: more than meets the eye. Genes & Dev 21:367-378.
Harvey A R, Hu Y, Leaver S G, Mellough C B, Park K, Verhaagen J, Plant G W, Cui Q (2006) Gene therapy and transplantation in CNS repair: The visual system. Prog Ret Eye Res 25:449-489.
Heanue T A, Pachnis V (2007) Enteric nervous system development and Hirschsprung's disease: advances in genetic and stem cell studies. Nat Rev Neurosci 8:466-479.
Henssler E M, Scholz O, Lochner S, Gmeiner P, Hillen W (2004) Structure-based design of Tet repressor to optimize a new inducer specificity. Biochemistry 43:9512-9518.
Hermann A, Maisel M, Storch A (2006) Epigenetic conversion of human adult bone mesodermal stromal cells into neuroectodermal cell types for replacement therapy of neurodegenerative disorders. Expert Opin Biol Ther 6:653-670.
Hevner R F, Hodge R D, Daza R A, Englund C (2006) Transcription factors in glutamatergic neurogenesis: conserved programs in neocortex, cerebellum, and adult hippocampus. Neurosci Res 55:223-233.
Hjerling-Leffier J, Marmigere F, Heglind M, Cederberg A, Koltzenburg M, Enerback S, Ernfors P (2005) The boundary cap: a source of neural crest stem cells that generate multiple sensory neuron subtypes (2005) Development 132:2623-2632.
Hoffman R M (2007) The potential of nestin-expressing hair follicle stem cells in regenerative medicine. Expert Opin Biol Ther 7:289-291.
Holland A M, Hale M A, Kagami H, Hammer R E, MacDonald R J (2002) Experimental control of pancreatic development and maintenance. PNAS 99:12236-12241.
Hong C S, Saint-Jeannet J P (2005) Sox proteins and neural crest development. Semin Cell Dev Biol 16:694-703.
Hsu Y C, Lee D C, Chiu I M (2007) Neural stem cells, neural progenitors, and neurotrophic factors. Cell Transplant 16:133-150.
Hu Z, Ulfendahl M (2006) Cell replacement therapy in the inner ear. Stem Cells Dev 15:449-459.
Huang E J, Reichardt L F (2003) Trk receptors: roles in neuronal signal transduction. Annu Rev Biochem 72:609-642.
Inoue K, Khajavi M, Ohyama T, Hirabayashi S, Wilson J, Reggin J D, Mancias P, Butler I J, Wilkinson M F, Wegner M, Lupski J R (2004) Molecular mechanism for distinct neurological phenoty pes conveyed by allelic truncating mutations. Nat Genet. 36:361-369.
Jankovic J, Chen S, Le W D (2005) The role of Nurr1 in the development of dopaminergic neurons and Parkinson's disease. Prog Neurobiol 77:128-138.
Kageyama R, Ohtsuka T, Hatakeyama J, Ohsawa R (2005) Roles of bHLH genes in neural stem cell differentiation. Exp Cell Res 306:343-348.
Kele J, Simplicio N, Ferri A L, Mira H, Guillemot F, Arenas E, Ang S L (2006) Neurogenin 2 is required for the development of ventral midbrain dopaminergic neurons. Development 133:495-505.
Kerrison J B, Duh E J, Yu Y, Otteson D C, Zack D J (2005) A system for inducible gene expression in retinal ganglion cells. Invest Ophthalmol Vis Sci 46:2932-2939.
Keyoung H M, Goldman S A (2007) Glial progenitor-based repair of demyelinating neurological diseases. Neurosurg Clin N Am 18:93-104.
Khlistunova I, Biernat J, Wang Y, Pickhardt M, von Bergen M, Gazova Z, Mandelkow E, Mandelkow E M (2006) Inducible expression of Tau repeat domain in cell models of tauopathy: aggregation is toxic to cells but can be reversed by inhibitor drugs. J Biol Chem 281:1205-1214.
Kholodilov N, Yarygina O, Oo T F, Zhang H, Sulzer D, Dauer W, Burke R E (2004) Regulation of the development of mesencephalic dopaminergic systems by the selective expression of glial cell line-derived neurotrophic factor in their targets. J Neurosci 24:3136-3146.
Kim J, Lo L, Dormand E, Anderson D J (2003) SOX10 maintains multipotency and inhibits neuronal differentiation of neural crest stem cells. Neuron 38:17-31.
Kramer B P, Weber W, Fussenegger M (2003) Artificial regulatory networks and cascades for discrete multilevel transgene control in mammalian cells. Biotechnol Bioeng 83:810-820.
Kyba M, Perlingeiro R C, Daley G Q (2002) HoxB4 confers definitive lymphoid-myeloid engraftment potential on embryonic stem cell and yolk sac hematopoietic progenitors. Cell 109:29-37.
Lee B, Kim J, Kim S J, Lee H, Chang J W (2007) Constitutive GABA expression via a recombinant adeno-associated virus consistently attenuates neuropathic pain. Biochem Biophys Res Commun 357:971-976.
Leimeroth R, Lobsiger C, Lussi A, Taylor V, Suter U, Sommer L (2002) Membrane-bound neuregulin1 type III actively promotes Schwann cell differentiation of multipotent Progenitor cells. Dev Biol 246:245-258.
Levanon D, Bettoun D, Harris-Cerruti C, Woolf E, Negreanu V, Eilam R, Bernstein Y, Goldenberg D, Xiao C, Fliegauf M, Kremer E, Otto F, Brenner O, Lev Tov A, Groner Y (2002) The Runx3 transcription factor regulates development and survival of TrkC dorsal root ganglia neurons. EMBO J. 21:3454-3463.
Levanon D, Brenner O, Negreanu V, Bettoun D, Woolf E, Eilam R, Lotem J, Gat U, Otto F, Speck N, Groner Y (2001) Spatial and temporal expression pattern of Runx3 (Aml2) and Runx1 (Aml1) indicates non-redundant functions during mouse embryogenesis. Mech Dev 109:413-417.
Levinsson A, Holmberg H, Schouenborg J, Seiger A, Aldskogius H, Kozlova E N (2000) Functional connections are established in the deafferented rat spinal cord by peripherally transplanted human embryonic sensory neurons. Eur J Neurosci 12:3589-3595.
Lindblom J, Johansson A, Holmgren A, Grandin E, Nedergard C, Fredriksson R, Schioth H B (2006) Increased mRNA levels of tyrosine hydroxylase and dopamine transporter in the VTA of male rats after chronic food restriction. Eur J Neurosci 23:180-186.
Lindvall O, Kokaia Z, Martinez-Serrano A (2004) Stem cell therapy for human neurodegenerative disorders-how to make it work. Nat Med 10 (Suppl) S42-50.
Liu X, Alexander V, Vijayachandra K, Bhogte E, Diamond I, Glick A (2001) Conditional epidermal expression of TGFbeta 1 blocks neonatal lethality but causes a reversible hyperplasia and alopecia. P roc Natl Acad Sol USA 98:9139-9144.
Longhi L, Zanier E R, Royo N, Stocchetti N, McIntosh T K (2005) Stem cell transplantation as a therapeutic strategy for traumatic brain injury. Transpl Immunol. 15:143-148
Ludwig A, Schlierf B, Schardt A, Nave K A, Wegner M (2004) Sox10-rtTA mouse line for tetracycline-inducible expression of transgenes in neural crest cells and oligodendrocytes. Genesis 40:171-175.
Ma Q, Fode C, Guillemot F, Anderson D J (1999) Neurogenin1 and neurogenin2 control two distinct waves of neurogenesis in developing dorsal root ganglia. Genes Dev 13:1717-1728.
Marmigere F, Ernfors P (2007) Specification and connectivity of neuronal subtypes in the sensory lineage. Nat Rev Neurosci 8:114-27.
Marmigere F, Montelius A, Wegner M, Groner Y, Reichardt L F, Ernfors P (2006) The Runx1/AML1 transcription factor selectively regulates development and survival of TrkA nociceptive sensory neurons. Nat Neurosci 9:180-187.
MacLaren R E, Pearson R A, MacNeil A, Douglas R H, Salt T E, Akimoto M, Swaroop A, Sowden J C, Ali R R (2006) Retinal repair by transplantation of photoreceptor precursors. Nature 444:203-207.
Mansuy I M, Winder D G, Moallem T M, Osman M, Mayford M, Hawkins R D, Kandel E R (1998) Inducible and reversible gene expression with the rtTA system for the study of memory. Neuron. 21:257-265.
McCloskey D T, Turnbull L, Swigart P M, Zambon A C, Turcato S, Joho S, Grossman W, Conklin B R, Simpson P C, Baker A J (2005) Cardiac transgenesis with the tetracycline transactivator changes myocardial function and gene expression. Physiol Genomics 22:118-126.
Molyneaux B J, Arlotta P, Menezes J R, Macklis J D (2007) Neuronal subtype specification in the cerebral cortex. Nat Rev Neurosci 8:427-437.
Morgan J E (2005) Stem cells to treat muscular dystrophies. Acta Myol 24:181-183.
Naylor L H (1999) Reporter gene technology: the future looks bright.Biochem Pharmacol 58:749-757.
Ohsawa K, Ohtsuka T, Kageyama K (2005) Mash1 and Math3 are required for development of branchiomotor neurons and maintenance of neural progenitors. J Neurosci 25:5857-5865.
Park C-H, Kang J S, Kim J-S, Chung S, Koh J-Y, Yoon E-H, Jo A Y, Chang M-C, Koh H-C, Hwang S J, Suh-Kim H, Lee Y-S, Kim K-S, Lee S-H (2006) Differential actions of the proneural genes encoding Mash1 and neurogenins in Nurr1-induced dopamine neuron differentiation. J Cell Sci 119:2310-2320.
Parker A M, Katz A J (2006) Adipose-derived stem cells for the regeneration of damaged tissues. Expert Opin Biol Ther 6:567-578.
Parras C M, Hunt C, Sugimori M, Nakafuku M, Rowitch D, Guillemot F (2007) The proneural gene Mash1 specifies an early population of telencephalic oligodendrocytes. J Neurosci 27:4233-4242.
Pfeifer K, Vroemen M, Caioni M, Aigner L, Bogdahn U, Heidner N (2006) Autologous adult rodent progenitor cell transplantation represents a feasible strategy to promote structural repair in the chronically injured spinal cord. Future Med 1:255-266.
Pillai A, Mansouri A, Behringer R, Westphal H, Goulding M (2007) Lhx1 and Lhx5 maintain the inhibitory-neurotransmitter status of interneurons in the dorsal spinal cord. Development 134:357-366.
Poitras L, Ghanem N, Hatch G, Ekker M (2007) The proneural determinant MASH1 regulates forebrain Dlx1/2 expression through the 112b intergenic enhancer, Development 134:1755-1765.
Ramakers C, Ruijter J M, Deprez R H, Moorman A F (2003) Assumption-free analysis of quantitative real-time polymerase chain reaction (PCR) data. Neurosci Lett 2003 339:62-66.
Ross S E, Greenberg M E, Stiles C D (2003) Basic helix-loop-helix factors in cortical development. Neuron 39:13-25.
Rubinchik S, Woraratanadharm J, Yu H, Dong J Y (2005) New complex Ad vectors incorporating both rtTA and tTS deliver tightly regulated transgene expression both in vitro and in vivo. Gene Ther 12:504-511.
Saini A, Nasser A S, Stewart C E (2006) Waste management—cytokines, growth factors and cachexia. Cytokine Growth Factor Rev 17:475-486
Schnappinger D, Schubert P, Pfleiderer K, Hillen W (1998) Determinants of protein-protein recognition by four helix bundles: changing the dimerization specificity of Tet repressor. EMBO J. 17:535-543.
Schroeder I S, Rolletschek A, Blyszczuk P, Kania G, Wobus A M (2006) Differentiation of mouse embryonic stem cells to insulin-producing cells Nat Protoc 1:495-507.
Schwarz J, Schwarz S C, Storch A (2006) Developmental perspectives on human midbrain-derived neural stem cells. Neurodegener Dis 3:45-49.
Sieber-Blum M, Grim M, Hu Y F, Szeder V (2004) Pluripotent neural crest stem cells in the adult hair follicle. Dev Dyn 231:258-269.
Silver J, Miller J H (2004) Regeneration beyond the glial scar. Nat Rev Neurosci 5:146-156.
Sommer L (2001) Context-dependent regulation of fate decisions in multipotent progenitor cells of the peripheral nervous system. Cell Tissue Res 211-216.
Sommer L (2005) Checkpoints of melanocyte stem cell development. Sci STKE 2005:pe42.
Sonntag K C, Simantov R, Kim K S, Isacson O (2004) Temporally induced Nurr1 can induce a non-neuronal dopa minergic cell type in embryonic stem cell differentiation. Eur J Neurosci 19:1141-1152.
Stanchina L, Baral V, Robert F, Pingault V, Lemort N, Pachnis V, Goossens M, Bondurand N (2006) Interactions between Sox10, Edn3 and Ednrb during enteric nervous system and melanocyte development. Dev Biol 295:232-249.
Stolt C C, Lommes P, Sock E, Chaboissier M C, Schedl A, Wegner M (2003) The Sox9 transcription factor determines glial fate choice in the developing spinal cord. Genes Dev. 17:1677-1689.
Sugaya K, Alvarez A, Marutle A, Kwak Y D, Choumkina E (2006) Stem cell strategies for Alzheimer's disease therapy. Panminerva Med 48:87-96.
Sugimori M, Nagao M, Bertrand N, Parras C M, Guillemot F, Nakafuku M (2007) Combinatorial actions of patterning and HLH transcription factors in the spatiotemporal control of neurogenesis and gliogenesis in the developing spinal cord. Development 134:1617-1629.
Terzis J K, Vekris M D, Soucacos P N (2004) Brachial plexus root avulsions. World J Surg 25:1049-1061.
Thuret S, Moon L D, Gage F H (2006) Therapeutic interventions after spinal cord injury. Nat Rev Neurosci 7:628-643. Erratum in: Nat Rev Neurosci 7:902, 2006.
Toniatti C, Bujard H, Cortese R, Gilberto G (2004) Gene therapy progress and prospects: transcription regulatory systems. Gene Ther 11:649-657.
Turnbull L, Zhou H Z, Swigart P M, Turcato S, Karliner J S, Conklin B R, Simpson P C, Baker A J (2006) Sustained preconditioning induced by cardiac transgenesis with the tetracycline transactivator. Am J Physiol Heart Circ Physiol 290:H1103-1109.
Uchida S, Sakai S, Furuichi T, Hosoda H, Toyota K, Ishii T, Kitamoto A, Sekine M, Koike K, Masushige S, Murphy G, Silva A J, Kida S. Tight regulation of transgene expression by tetracycline-dependent activator and repressor in brain. Genes Brain Behav. 2006 Feb; 5(1):96-106.
Urlinger S, Baron U, Thellmann M, Hasan M T, Bujard H, Hillen W (2000) Exploring the sequence space for tetracycline-dependent transcriptional activators: novel mutations yield expanded range and sensitivity. PNAS 97:7963-7968
Valencia T G, Roberts L D, Zeng H, Grant S R (2000) Tetracycline-inducible CaM kinase II silences hypertrophy-sensitive gene expression in rat neonate cardiomyocytes. Biochem Biophys Res Commun 274:803-810.
Valencik M L, McDonald J A (2001) Codon optimization markedly improves doxycycline regulated gene expression in the mouse heart. Transgenic Res 10:269-275
Weber W, Fussenegger M (2006) Pharmacologaic transgene control systems for gene therapy. J Gene Med 8:535-556.
Weber W, Rimann M, Spielmann M, Keller B, Daoud-El Baba M, Aubel D, Weber C C, Fussenegger M (2004) Gas-inducible transgene expression in mammalian cells and mice. Nat Biotechnol 22:1440-1444.
Wolfe S Q, Garg M, Cumberbatch N M, Furst C, Martinez M, Hernandez M, Reimers R, Y, Gomez-Marin O, Eaton M J (2007) Optimizing the transplant dose of a human neuronal cell line graft to treat SCI pain in the rat. Neurosci Lett 414:121-125.
Wu S S, Frucht S J (2005) Treatment of Parkinson's disease: what's on the horizon? CNS Drugs 19:723-743.
Wutz A, Jaenisch R (2000) A shift from reversible to irreversible X inactivation is triggered during ES cell differentiation. Mol Cell 5:695-705.
Yan R-T, Ma M, Liang L, Wang S-Z (2005) bHLH genes and retinal cell fate specification. Mol Neurobiol 32:157-171.
Ye L, Chan S, Chow Y H, Tsui L C, Hu J (2001) Regulated expression of the human CFTR gene in epithelial cells. Mol Ther 3:723-733.
Young M J (2006) Stem cells in the mammalian eye: a tool for retinal repair. APMIS 113:845-857.

1. A medicament in two or more parts for cell differentiation to alleviate cell and cell-related deficiencies in a mammal, comprising in vitro produced non-activated inducible gene construct(s) capable of expressing transcription factor(s), and optionally additional suppressor(s) or activator(s) of expression of said transcription factor(s), in a cell for transfection, and separate exogenous inducer(s) for in vivo expression.

2. The medicament according to claim 1, wherein the cell for transfection is chosen from the group consisting of regional stem cells embryonic stem (ES) cells, neural crest stem cells, neural stem cells from brain and spinal cord, mesenchymal stem cells, endothelial stem cells, endodermal stem cells.

3. The medicament according to claim 1, wherein the transcription factor(s) are chosen from the group consisting of embryonic stem cell transcription factors NANOG, OCT3/4, and SOX2; neural crest stem cell transcription factors Brn3a, FoxD3, GATA-3, Hand2, Mash1, Mitf, Nanog, Ngn1/2, Oct-4, Pax3, Phox2a/b, Runx1/3, Slug, Sox4/8/9/10/11; neural stem cells from brain and spinal cord transcription factors Brn3a, Cash1, Cdx2, Dbx1/2, Dlx1/2, Ebf1, Emx1/2, En1/2, ER81, Evx1, Foxg1, Foxp2, Gbx2, Gli2/3, Gsh1/2, Hb9, Hes1/5, Hoxb4/9, Id1/3, Islet1/2, Lhx1/2/5/6/7, LIM1/2/3, Lmx1a, Mash1, Math1, MNR2, Msx1, NeuroD, Ngn1/2/3, Nkx2.1/2, Nkx6.1/2, Olig1/2/3, Otx1/2, Pax3/5/6/7/8, Phox2b, Pitx3, Prox1, Pff1, ROR, Sim1, Sox10, Tbr2, Tlx-3, Trb1; mesenchymal stem cell transcription factors C/EBP-alpha, Dlx5, Fli1, Gata2/3/4, Gli3, KROX20, Msx1/2, Myf5, MyoD, Oct-4, Pax3/6/7, PPAR-gamma, Runx2, Rex-1, Sox2/9/10; endothelial stem cell transcription factors ARNT, ELF-1, EPAS, Ets1, Fra1, GATA2/3, gridlock, HIF-1alpha, HOXD3, LMO2, NERF-2, Prox-1, Runx1, Scl, Sox2, Sp1/3, TBP, Vezf1, YY1; endodermal stem cell transcription factors Beta2/NeuroD, FoxD3, Hex, Hnf3/6, Hlxb9, Islet1, MafA, Meis2, Ngn3, Nkx2.2, Nkx6.1, Oct-4, Pax4/6, Pbx1, Pdx1.

4. The medicament according to claim 1, wherein the cell and cell-related deficiencies in a mammal is a disease chosen from the group consisting of, traumatic injuries to the brain and spinal cord, neurodegenerative disorders, stroke, demyelinating disorders, neuropathic pain disorders, diabetes, myocardial infarction, skeletal muscle disorders.

5. The medicament according to claim 1, wherein the inducible gene construct(s) comprise(s) conditional and compatible transcription control systems which are able to provide simultaneous or independent activity of transgene(s) to regulate stem cell differentiation in response to exogenous inducer(s).

6. The medicament according to claim 5, wherein the exogenous inducer(s) are chosen from the group consisting of tetracycline, streptogramin and macrolide.

7. A delivery system for delivery of a medicament in two or more parts to a mammal, comprising as a first part non-activated inducible gene construct(s) capable of expressing transcription factor(s), and optionally additional suppressor(s) or activator(s) of expression of said transcription factor(s), in a cell, and as a second part exogenous inducer(s) of expression, the delivery of the second part being after or simultaneously with the delivery of the first part.

8. A method of treating cell and cell-related deficiencies in a mammal, comprising the consecutive steps:
(a) transfection of cells with non-activated inducible gene construct(s) capable of expressing transcription factor(s), and optionally additional suppressor(s) or activator(s) of expression of said transcription factor(s), in a cell;
(b) transplantation of said cells to said mammal, and
(c) activation or suppression of said inducible gene construct(s) by administration of exogenous inducer(s) to said mammal for expression of said transcription factors in cells of said mammal.

9. The method according to claim 8, wherein the cells for transfection are chosen from the group consisting of regional stem cells, embryonic (ES) stem cells, neural crest stem cells, neural stem cells from brain and spinal cord, mesenchymal stem cells, endothelial stem cells, endodermal stem cells; and the transcription factors are chosen from the group consisting of embryonic stem cell transcription factors NANOG, OCT3/4, and Sox2; neural crest stem cell transcription factors Brn3a, FoxD3, GATA-3, Hand2, Mash1, Mitf, Nanog, Ngn1/2, Oct-4, Pax3, Phox2a/b, Runx1/3, Slug, Sox4/8/9/10/11; neural stem cells from brain and spinal cord transcription factors Brn3a, Cash1, Cdx2, Dbx1/2, Dlx1/2, Ebf1, Emx1/2, En1/2, ER81, Evx1, Foxg1, Foxp2, Gbx2, Gli2/3, Gsh1/2, Hb9, Hes1/5, Hoxb4/9, Id1/3, Islet1/2, Lhx1/2/5/6/7, LIM1/2/3, Lmx1a, Mash1, Math1, MNR2, Msx1, NeuroD, Ngn1/2/3, Nkx2.1/2, Nkx6.1/2, Olig1/2/3, Otx1/2, Pax3/5/6/7/8, Phox2b, Pitx3, Prox1, Pff1, ROR, Sim1, Sox10, Tbr2, Tlx-3, Trb1; mesenchymal stem cell transcription factors C/EBP-alpha, Dlx5, Fli1, Gata2/3/4, Gli3, KROX20, Msx1/2, Myf5, MyoD, Oct-4, Pax3/6/7, PPAR-gamma, Runx2, Rex-1, Sox2/9/10; endothelial stem cell transcription factors ARNT, ELF-1, EPAS, Ets1, Fra1, GATA2/3, gridlock, HIF-1alpha, HOXD3, LMO2, NERF-2, Prox-1, Runx1, Scl, Sox2, Sp1/3, TBP, Vezf1, YY1; endodermal stem cell transcription factors Beta2/NeuroD, FoxD3, Hex, Hnf3/6, Hlxb9, Islet1, MafA, Meis2, Ngn3, Nkx2.2, Nkx6.1, Oct-4, Pax4/6, Pbx1, Pdx1.


SEQUENCE LISTING (42 sequences)

SEQ ID NO: 1 - DNA, 1120 bp, Unknown
  Mouse - Rosa26-rtTA
gacctccatagaagacaccgggaccgatccagcctccgcggccccgaattcaccatgccaaagagacccagaccctctagattagataaaagtaaagtgattaacagcgcattagagctgcttaatgaggtcggaatcgaaggtttaacaacccgtaaactcgcccagaagcttggtgtagagcagcctacactgtattggcatgtaaaaaataagcgggctttgctcgacgccttagccattgagatgttagataggcaccatactcacttttgccctttaaaaggggaaagctggcaagattttttacgcaataacgctaaaagttttagatgtgctttactaagtcatcgcaatggagcaaaagtacattcagatacacggcctacagaaaaacagtatgaaactctcgaaaatcaattagcctttttatgccaacaaggtttttcactagagaacgcgttatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgag

SEQ ID NO: 2 - DNA, 740 bp, Unknown
  Mouse - eYFP-TREbi-Aml1A/Runx1
caccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccctagagctc

SEQ ID NO: 3 - DNA, 1347 bp, Unknown
  Mouse - Rosa26-rtTA-HPRT-Aml1A-IRES-eGFP
cgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggaggaagacacagcaccctggagatgttaaggcagaagtcagttcttcttccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 4 - DNA, 747 bp, Unknown
  Mouse - Sox10-rtTAS-M2
atgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 5 - DNA, 6693 bp, Artificial Sequence
  Plasmid pBi-EYFP-FLAG-Aml1B
accggggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgtgctagcgcggccgctctaggcctaggggataacttcgtatagcatacattatacgaagttattctagcctaccatggcagactacaaggacgacgatgacaagcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccatcgatccggataacttcgtatagcatacattatacgaagttatcctaggggaattcgatatcaagcttatcgataagcttgtcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcactcgacgctggtcgagctgatacttcccgtccgccaggggacatgccggcgatgctgaaggtcgcgcgcattcccgatgaagaggccggttaccgcctgttgacctggtgggacgggcagggcgccgcccgagtcttcgcctcggcggcgggcgctctgctcatggagcgcgcgtccggggccggggaccttgcacagatagcgtggtccggccaggacgacgaggcttgcaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcctctagaactagttctagtcgagaagttcctatactttctagagaataggaacttcggaataggaacttcacgcgtgaattgccgctttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcg

SEQ ID NO: 6 - DNA, 6108 bp, Artificial Sequence
  Plasmid pBi-EYFP-HA-Aml1A
accggggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgtgctagcgcggccgctctaggcctaggggataacttcgtatagcatacattatacgaagttattctagcctctagaccatggcatacccatacgacgtgcctgactacgcctcccgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggaggaagacacagcaccctggagatgttaaggcagaagtcagttcttctatcggatccggataacttcgtatagcatacattatacgaagttatcctaggggaattcgatatcaagcttatcgataagcttgtcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcactcgacgctggtcgagctgatacttcccgtccgccaggggacatgccggcgatgctgaaggtcgcgcgcattcccgatgaagaggccggttaccgcctgttgacctggtgggacgggcagggcgccgcccgagtcttcgcctcggcggcgggcgctctgctcatggagcgcgcgtccggggccggggaccttgcacagatagcgtggtccggccaggacgacgaggcttgcaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcctctagaactagttctagtcgagaagttcctatactttctagagaataggaacttcggaataggaacttcacgcgtgaattgccgctttacttgtacagctcgtccatgccgagagtgatcccggcggcggtcacgaactccagcaggaccatgtgatcgcgcttctcgttggggtctttgctcagggcggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcg

SEQ ID NO: 7 - DNA, 4035 bp, Artificial Sequence
  Plasmid pWHE120 (sM2) -M3
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaagtcataaacggcgctctggaattactcaatggagtcggtatcgaaggcctgacgacaaggaaactcgctcaaaagctgggattcgcccagaagaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 4038 bp, Artificial Sequence
  Plasmid pWHE120 (B/D) -2.1
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagattagataaaagtaaagtgattaacagcgcattagagctgcttaatgaggtcggaatcgaaggtttaacaacccgtaaactcgcccagaagcttggtgtagagcagcctacattgtattggcatgtaaaaaataagcgggccctactggatgcgctggcggtggagatcttggcgcgtcataaggattattcactgcctgcggcgggggaatcctggcagtcatttctgcgcaataatgcaatgagtttccgccgcgcgctgctgcgttaccgtgacggggcaaaagtgcacctcggcacgcgtcctgatgaaaaacagtatgatacggtggaaacccagttacgctttatgacagaaaacggcttttcactgcgcgacgggttatatgcgattttagcggtcatccattttactttaggtgccgtactggagcagcaggagcatactgccgccctgaccgaccgccctgcagcaccggacgaaaacctgccgccgctattgcgggaagcgctgcagattatggacagtgatgatggtgagcaggcctttctgcatggcctggagagcctgatccgggggtttgaggtgcagcttacggcactgttgcaaatagtgccggccgacgcccttgacgattttgacttagacatgctcccagccgatgcccttgacgactttgaccttgatatgctgcctgctgacgctcttgacgattttgaccttgacatgctccccgggtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 3900 bp, Artificial Sequence
  Plasmid pWHE1145
ctcgagtttactccccgtcagtgacggagaacgtatgtcgagtttactccccgtcagtgacggagaacgtatgtcgagtttactccccgtcagtgacggagaacgtatgtcgagtttactccccgtcagtgacggagaacgtatgtcgagtttactccccgtcagtgacggagaacgtatgtcgagtttactccccgtcagtgacggagaacgtatgtcgagtttactccccgtcagtgacggagaacgtatgtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcaagatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctagaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 21 bp, Artificial Sequence
  Primer Sox10-F
ctaggctgtcagagcagacga

SEQ ID NO: 11 - DNA, 21 bp, Artificial Sequence
  Primer Sox10-R
ctccacctctgataggtcttg

SEQ ID NO: 12 - DNA, 21 bp, Artificial Sequence
  Primer rtTA2S-M2-R
ctcgattggcagggcatcgag

SEQ ID NO: 13 - DNA, 20 bp, Artificial Sequence
  Primer “rtTA-F
cgcaatggagcaaaagtaca

SEQ ID NO: 14 - DNA, 20 bp, Artificial Sequence
  Primer rtTA-R
cctcgatggtagacccgtaa

SEQ ID NO: 15 - DNA, 20 bp, Artificial Sequence
  Primer rtTA-R complementary sequence
ttacgggtctaccatcgagg

SEQ ID NO: 16 - DNA, 20 bp, Artificial Sequence
  Primer eYFP-F
gacgtaaacggccacaagtt

SEQ ID NO: 17 - DNA, 20 bp, Artificial Sequence
  Primer eYFP-R
gtcctccttgaagtcgatgc

SEQ ID NO: 18 - DNA, 20 bp, Artificial Sequence
  Primer eYFP-R complementary sequence
gcatcgacttcaaggaggac

SEQ ID NO: 19 - DNA, 20 bp, Artificial Sequence
  Primer Aml1A/Runx1-F
aaccctcagcctcagagtca

SEQ ID NO: 20 - DNA, 20 bp, Artificial Sequence
  Primer IRES-R
aggaactgcttccttcacga

SEQ ID NO: 21 - DNA, 20 bp, Artificial Sequence
  Primer Sox10.F
tctacactgccatctctgac

SEQ ID NO: 22 - DNA, 17 bp, Artificial Sequence
  Primer Brn3a-2-F
ctcctccactgccaagc

SEQ ID NO: 23 - DNA, 22 bp, Artificial Sequence
  Primer Brn3a-2-F
aggcctattttgccgtacaacc

SEQ ID NO: 24 - DNA, 28 bp, Artificial Sequence
  Primer Brn3a-2-R
ctcctcagtaagtggcagagaatttcat

SEQ ID NO: 25 - DNA, 17 bp, Artificial Sequence
  Primer Krox20-forw
tcctccagcgtcacctc

SEQ ID NO: 26 - DNA, 17 bp, Artificial Sequence
  Primer Krox20-rev
ggcggcgataagaatgc

SEQ ID NO: 27 - DNA, 20 bp, Artificial Sequence
  Primer Foxs1-F
ggccctgtcaagatagacca

SEQ ID NO: 28 - DNA, 20 bp, Artificial Sequence
  Primer Foxs1-R
actttgccgagcacatctct

SEQ ID NO: 29 - DNA, 21 bp, Artificial Sequence
  Primer Neurogenin1-forw
ccagtagtccctcggcttcag

SEQ ID NO: 30 - DNA, 21 bp, Artificial Sequence
  Primer Neurogenin1-rev
gtcgtgtggagcaggtctttg

SEQ ID NO: 31 - DNA, 18 bp, Artificial Sequence
  Primer Neurogenin2-forw
cggagaagcatcgttatg

SEQ ID NO: 32 - DNA, 17 bp, Artificial Sequence
  Primer Neurogenin2-rev
gggagaaggatgggaag

SEQ ID NO: 33 - DNA, 17 bp, Artificial Sequence
  ctctgctccg tgctacc
ctctgctccgtgctacc

SEQ ID NO: 34 - DNA, 19 bp, Artificial Sequence
  Primer Aml1A/Runx1-rev
gtcgttgaatctcgctacc

SEQ ID NO: 35 - DNA, 17 bp, Artificial Sequence
  Primer hAml1A-forw
cctctgctccgtgctgc

SEQ ID NO: 36 - DNA, 20 bp, Artificial Sequence
  Primer hAml1A-rev
ggtcattaaatcttgcaacc

SEQ ID NO: 37 - DNA, 20 bp, Artificial Sequence
  Primer Aml1A/Runx1-F
aaccctcagcctcagagtca

SEQ ID NO: 38 - DNA, 20 bp, Artificial Sequence
  Primer IRES-R
aggaactgcttccttcacga

SEQ ID NO: 39 - DNA, 17 bp, Artificial Sequence
  Primer Runx3-forw
ttcacgctcacaatcac

SEQ ID NO: 40 - DNA, 19 bp, Artificial Sequence
  Primer Runx3-rev
ctggtcttctatcttctgc

SEQ ID NO: 41 - DNA, 21 bp, Artificial Sequence
  Primer Mts1/S100A4-forw
acagcaacagggacaatgaag

SEQ ID NO: 42 - DNA, 20 bp, Artificial Sequence
  Primer Mts1/S100A4-rev
gcgaagaagccagagtaagg